SEQUENCE LISTING (40 sequences)

SEQ ID NO: 1 - protein, 355 aa, Homo sapiens
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 2 - protein, 358 aa, Sus scrofa
AEMKTYSQRAVANACALVMCSPMENGLPGRDGRDGREGPRGEKGDPGLPGAVGRAGMPGLAGPVGPKGDNGSTGEPGAKGDIGPCGPPGPPGIPGPAGKEGPSGQQGNIGPPGTPGPKGETGPKGEVGALGMQGSTGARGPAGLKGERGAPGERGAPGSAGAAGPAGATGPQGPSGARGPPGLKGDRGPPGERGAKGESGLPGITALRQQVETLQGQVQRLQKAFSQYKKVELFPNGRGVGEKIFKTGGFEKTFQDAQQVCTQAGGQMASPRSETENEALSQLVTAQNKAAFLSMTDIKTEGNFTYPTGEPLVYANWAPGEPNNNGGSSGAENCVEIFPNGKWNDKACGELRLVICEF

SEQ ID NO: 3 - protein, 358 aa, Artificial Sequence
  Synthetic
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFEKPFTEAQLLCTQAGGQLASPRSAAENAALQQLVVAQNKAAFLSMTDSKTEGKFTYPTGESLVYSNWAPGEPNNDXXXXGSENCVEIFTNGKWNDRACGEKRLVVCEF

SEQ ID NO: 4 - protein, 358 aa, Artificial Sequence
  Synthetic
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFEKPFTEAQLLCTQAGGQLASPRSAAENAALQQLVVAQNKAAFLSMTDSKTEGKFTYPTGESLVYSNWAPGEPNNDXXXXGSEDCVEIFTNGKWNDRACGEKRLVVCEF

SEQ ID NO: 5 - protein, 358 aa, Artificial Sequence
  Synthetic
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFEKPFTEAQLLCTQAGGQLASPRSAAENAALQQLVVAQNKAAFLSMTDSKTEGKFTYPTGESLVYSNWAPGEPNDDXXXXGSEDCVEIFTNGKWNDRACGEKRLVVCEF

SEQ ID NO: 6 - protein, 358 aa, Artificial Sequence
  Synthetic
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNNDXXXXGSENCVEIFTNGKWNDRACGEKRLVVCEF

SEQ ID NO: 7 - protein, 358 aa, Artificial Sequence
  Synthetic
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDXXXXGSENCVEIFTNGKWNDRACGEKRLVVCEF

SEQ ID NO: 8 - protein, 358 aa, Artificial Sequence
  Synthetic
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDXXXXGSEDCVEIFTNGKWNDRACGEKRLVVCEF

SEQ ID NO: 9 - protein, 355 aa, Artificial Sequence
  Synthetic
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFEKPFTEAQLLCTQAGGQLASPRSAAENAALQQLVVAQNKAAFLSMTDSKTEGKFTYPTGESLVYSNWAPGEPNDDGGSEDCVEIFTNGKWNDRACGEKRLVVCEF

SEQ ID NO: 10 - protein, 355 aa, Artificial Sequence
  Synthetic
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFEKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 11 - protein, 358 aa, Artificial Sequence; identical to SEQ ID NO: 8
  Synthetic

SEQ ID NO: 12 - protein, 358 aa, Artificial Sequence
  Synthetic
AEMKTYSHRTTPSACTLVMCSSVESGLPGRDGRDGREGPRGEKGDPGLPGAAGQAGMPGQAGPVGPKGDNGSVGEPGPKGDTGPSGPPGPPGVPGPAGREGPLGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGXXXXEDCVEIFTNGKWNDRACGEKRLVVCEF

SEQ ID NO: 13 - DNA, 1726 bp, Homo sapiens
cggaattagggagatagttggtattaggattaggattgttgtgaagtatagtacggatgctacttgtgcaatgatggtaaaagggtagcttactggttgtcctccgattcaggttagaatgaggaggtctgcggcttggagctcctggggcctaacaaaaagaaacctgccatgctgctcttcctcctctctgcactggtcctactcacacagcccctgggctacctggaagcagaaatgaagacctactcccacagaacaatgcccagtgcttgcaccctggtcatgtgtagctcagtggagagtggcctgcctggtcgcgatggacgggatgggagagagggccctcggggcgagaagggggacccaggtttgccaggagctgcagggcaagcagggatgcctggacaagctggcccagttgggcccaaaggggacaatggctctgttggagaacctggaccaaagggagacactgggccaagtggacctccaggacctcccggtgtgcctggtccagctggaagagaaggtcccctggggaagcaggggaacataggacctcagggcaagccaggcccaaaaggagaagctgggcccaaaggagaagtaggtgccccaggcatgcagggctcggcaggggcaagaggcctcgcaggccctaagggagagcgaggtgtccctggtgagcgtggagtccctggaaacacaggggcagcagggtctgctggagccatgggtccccagggaagtccaggtgccaggggacccccgggattgaagggggacaaaggcattcctggagacaaaggagcaaagggagaaagtgggcttccagatgttgcttctctgaggcagcaggttgaggccttacagggacaagtacagcacctccaggctgctttctctcagtataagaaagttgagctcttcccaaatggccaaagtgtcggggagaagattttcaagacagcaggctttgtaaaaccatttacggaggcacagctgctgtgcacacaggctggtggacagttggcctctccacgctctgccgctgagaatgccgccttgcaacagctggtcgtagctaagaacgaggctgctttcctgagcatgactgattccaagacagagggcaagttcacctaccccacaggagagtccctggtctattccaactgggccccaggggagcccaacgatgatggcgggtcagaggactgtgtggagatcttcaccaatggcaagtggaatgacagggcttgtggagaaaagcgtcttgtggtctgcgagttctgagccaactggggtgggtggggcagtgcttggcccaggagtttggccagaagtcaaggcttagaccctcatgctgccaatatcctaataaaaaggtgaccatcaaaaaaaaaagctgagaatgccgccttgcaacagctggtcgtagctaagaacgaggctgctttcctgagcatgactgattccaagacagagggcaagttcacctaccccacaggagagtccctggtctattccaactgggccccaggggagcccaacgatgatggcgggtcagaggactgtgtggagatcttcaccaatggcaagtggaatgacagggcttgtggagaaaagcgtcttgtggtctgcgagttctgagccaactggggtgggtggggcagtgcttggcccaggagtttggccagaagtcaaggcttagaccctcatg

SEQ ID NO: 14 - DNA, 1385 bp, Sus scrofa
cgagtttgcctggagattctgagctctagaggacgcaactgacatgcttctcctccctctctccgtgctgatcctgctcacacagcccccgaggtcactgggagcagaaatgaagacctattcccagagagcagtggccaacgcctgcgccctggtcatgtgtagccccatggagaatggcctgcctggtcgtgatggtcgggatgggagagagggccctcggggcgagaagggggatccaggtttgccaggagctgtagggcgagcggggatgcctggactggctggcccagttgggcccaaaggggacaacggctctactggagaacccggagcaaagggagacattggaccatgcgggcctccaggacctccaggtatacctggtccagccggaaaagaaggtccctcagggcagcaggggaacataggacctccaggcacaccaggccccaaaggagagactgggcccaaaggagaagtgggtgccctgggcatgcagggctctacaggggcaagaggccctgcaggtcttaaaggagagagaggtgcccccggtgagcgtggagcccctggaagtgctggggcagcagggcctgctggagccacgggccctcagggcccttcaggtgccaggggccccccaggactgaaaggggacagaggtcctcctggagaaagaggagccaagggagagagtggactcccaggcatcactgctctgaggcaacaggtggagaccttacaggggcaggtacaacgcctccagaaggccttctctcagtataagaaagtggagctcttccccaatggccgaggtgtcggggagaagatcttcaagacgggaggctttgaaaagacttttcaggatgctcagcaggtatgcacacaggccgggggacagatggcctccccacgctctgagactgagaacgaggccttgagccagctggtcacagctcagaataaggctgctttcctgagcatgactgacatcaagacggagggcaatttcacctaccccacgggggagcccctggtctatgccaactgggcccctggggagcccaacaacaatggtggcagcagcggagcagagaactgtgtggagatctttcccaatggcaagtggaatgacaaggcctgcggagaactgcgcctcgtgatctgcgagttctgagcccctggggagggaggggcggtgtccagagctgtgtgctaccaacgtcccaataaataggtgaccttctgctggccagggcttctccacagagccgtgggacgaggccagaaggtagggagcctatggaacgcctccctcagaataaagtacgaaactggcctcacaaaaaaaaaaaaaaaaaaaaaaaaaaaaaaaaaaaaaa

SEQ ID NO: 15 - DNA, 1225 bp, Artificial Sequence
  Synthetic
aacaaaaagaaacctgccatgctgctcttcctcctctctgcactggtcctactcacacagcccctgggctacctggaagcagaaatgaagacctactcccacagaacaatgcccagtgcttgcaccctggtcatgtgtagctcagtggagagtggcctgcctggtcgcgatggacgggatgggagagagggccctcggggcgagaagggggacccaggtttgccaggagctgcagggcaagcagggatgcctggacaagctggcccagttgggcccaaaggggacaatggctctgttggagaacctggaccaaagggagacactgggccaagtggacctccaggacctcccggtgtgcctggtccagctggaagagaaggtcccctggggaagcaggggaacataggacctcagggcaagccaggcccaaaaggagaagctgggcccaaaggagaagtaggtgccccaggcatgcagggctcggcaggggcaagaggcctcgcaggccctaagggagagcgaggtgtccctggtgagcgtggagtccctggaaacacaggggcagcagggtctgctggagccatgggtccccagggaagtccaggtgccaggggacccccgggattgaagggggacaaaggcattcctggagacaaaggagcaaagggagaaagtgggcttccagatgttgcttctctgaggcagcaggttgaggccttacagggacaagtacagcacctccaggctgctttctctcagtataagaaagttgagctcttcccaaatggccaaagtgtcggggagaagattttcaagacagcaggctttgaaaaaccatttacggaggcacagctgctgtgcacacaggctggtggacagttggcctctccacgctctgccgctgagaatgccgccttgcaacagctggtcgtagctcagaacaaggctgctttcctgagcatgactgattccaagacagagggcaagttcacctaccccacaggagagtccctggtctattccaactgggccccaggggagcccaacaatgatggcggcagcagcgggtcagagaactgtgtggagatcttcaccaatggcaagtggaatgacagggcttgtggagaaaagcgtcttgtggtctgcgagttctgagccaactggggtgggtggggcagtgcttggcccaggagtttggccagaagtcaaggcttagaccctcatg

SEQ ID NO: 16 - DNA, 1074 bp, Artificial Sequence
  Synthetic
gcggaaatgaaaacctatagccatcgcaccaccccgagcgcgtgcaccctggtgatgtgcagcagcgtggaaagcggcctgccgggccgcgatggccgcgatggccgcgaaggcccgcgcggcgaaaaaggcgatccgggcctgccgggcgcggcgggccaggcgggcatgccgggccaggcgggcccggtgggcccgaaaggcgataacggcagcgtgggcgaaccgggcccgaaaggcgataccggcccgagcggcccgccgggcccgccgggcgtgccgggcccggcgggccgcgaaggcccgctgggcaaacagggcaacattggcccgcagggcaaaccgggcccgaaaggcgaagcgggcccgaaaggcgaagtgggcgcgccgggcatgcagggcagcgcgggcgcgcgcggcctggcgggcccgaaaggcgaacgcggcgtgccgggcgaacgcggcgtgccgggcaacaccggcgcggcgggcagcgcgggcgcgatgggcccgcagggcagcccgggcgcgcgcggcccgccgggcctgaaaggcgataaaggcattccgggcgataaaggcgcgaaaggcgaaagcggcctgccggatgtggcgagcctgcgccagcaggtggaagcgctgcagggccaggtgcagcatctgcaggcggcgtttagccagtataaaaaagtggaactgtttccgaacggccagagcgtgggcgaaaaaatttttaaaaccgcgggctttgaaaaaccgtttaccgaagcgcagctgctgtgcacccaggcgggcggccagctggcgagcccgcgcagcgcggcggaaaacgcggcgctgcagcagctggtggtggcgcagaacaaagcggcgtttctgagcatgaccgatagcaaaaccgaaggcaaatttacctatccgaccggcgaaagcctggtgtatagcaactgggcgccgggcgaaccgaacaacgatggcggcagcagcggcagcgaagattgcgtggaaatttttaccaacggcaaatggaacgatcgcgcgtgcggcgaaaaacgcctggtggtgtgcgaattt

SEQ ID NO: 17 - DNA, 1074 bp, Artificial Sequence
  Synthetic
gcggaaatgaaaacctatagccatcgcaccaccccgagcgcgtgcaccctggtgatgtgcagcagcgtggaaagcggcctgccgggccgcgatggccgcgatggccgcgaaggcccgcgcggcgaaaaaggcgatccgggcctgccgggcgcggcgggccaggcgggcatgccgggccaggcgggcccggtgggcccgaaaggcgataacggcagcgtgggcgaaccgggcccgaaaggcgataccggcccgagcggcccgccgggcccgccgggcgtgccgggcccggcgggccgcgaaggcccgctgggcaaacagggcaacattggcccgcagggcaaaccgggcccgaaaggcgaagcgggcccgaaaggcgaagtgggcgcgccgggcatgcagggcagcgcgggcgcgcgcggcctggcgggcccgaaaggcgaacgcggcgtgccgggcgaacgcggcgtgccgggcaacaccggcgcggcgggcagcgcgggcgcgatgggcccgcagggcagcccgggcgcgcgcggcccgccgggcctgaaaggcgataaaggcattccgggcgataaaggcgcgaaaggcgaaagcggcctgccggatgtggcgagcctgcgccagcaggtggaagcgctgcagggccaggtgcagcatctgcaggcggcgtttagccagtataaaaaagtggaactgtttccgaacggccagagcgtgggcgaaaaaatttttaaaaccgcgggctttgaaaaaccgtttaccgaagcgcagctgctgtgcacccaggcgggcggccagctggcgagcccgcgcagcgcggcggaaaacgcggcgctgcagcagctggtggtggcgcagaacaaagcggcgtttctgagcatgaccgatagcaaaaccgaaggcaaatttacctatccgaccggcgaaagcctggtgtatagcaactgggcgccgggcgaaccgaacgatgatggcggcagcagcggcagcgaagattgcgtggaaatttttaccaacggcaaatggaacgatcgcgcgtgcggcgaaaaacgcctggtggtgtgcgaattt

SEQ ID NO: 18 - DNA, 1074 bp, Artificial Sequence
  Synthetic
gcggaaatgaaaacctatagccatcgcaccaccccgagcgcgtgcaccctggtgatgtgcagcagcgtggaaagcggcctgccgggccgcgatggccgcgatggccgcgaaggcccgcgcggcgaaaaaggcgatccgggcctgccgggcgcggcgggccaggcgggcatgccgggccaggcgggcccggtgggcccgaaaggcgataacggcagcgtgggcgaaccgggcccgaaaggcgataccggcccgagcggcccgccgggcccgccgggcgtgccgggcccggcgggccgcgaaggcccgctgggcaaacagggcaacattggcccgcagggcaaaccgggcccgaaaggcgaagcgggcccgaaaggcgaagtgggcgcgccgggcatgcagggcagcgcgggcgcgcgcggcctggcgggcccgaaaggcgaacgcggcgtgccgggcgaacgcggcgtgccgggcaacaccggcgcggcgggcagcgcgggcgcgatgggcccgcagggcagcccgggcgcgcgcggcccgccgggcctgaaaggcgataaaggcattccgggcgataaaggcgcgaaaggcgaaagcggcctgccggatgtggcgagcctgcgccagcaggtggaagcgctgcagggccaggtgcagcatctgcaggcggcgtttagccagtataaaaaagtggaactgtttccgaacggccagagcgtgggcgaaaaaatttttaaaaccgcgggctttgtgaaaccgtttaccgaagcgcagctgctgtgcacccaggcgggcggccagctggcgagcccgcgcagcgcggcggaaaacgcggcgctgcagcagctggtggtggcgaaaaacgaagcggcgtttctgagcatgaccgatagcaaaaccgaaggcaaatttacctatccgaccggcgaaagcctggtgtatagcaactgggcgccgggcgaaccgaacaacgatggcggcagcagcggcagcgaaaactgcgtggaaatttttaccaacggcaaatggaacgatcgcgcgtgcggcgaaaaacgcctggtggtgtgcgaattt

SEQ ID NO: 19 - DNA, 1074 bp, Artificial Sequence
  Synthetic
gcggaaatgaaaacctatagccatcgcaccaccccgagcgcgtgcaccctggtgatgtgcagcagcgtggaaagcggcctgccgggccgcgatggccgcgatggccgcgaaggcccgcgcggcgaaaaaggcgatccgggcctgccgggcgcggcgggccaggcgggcatgccgggccaggcgggcccggtgggcccgaaaggcgataacggcagcgtgggcgaaccgggcccgaaaggcgataccggcccgagcggcccgccgggcccgccgggcgtgccgggcccggcgggccgcgaaggcccgctgggcaaacagggcaacattggcccgcagggcaaaccgggcccgaaaggcgaagcgggcccgaaaggcgaagtgggcgcgccgggcatgcagggcagcgcgggcgcgcgcggcctggcgggcccgaaaggcgaacgcggcgtgccgggcgaacgcggcgtgccgggcaacaccggcgcggcgggcagcgcgggcgcgatgggcccgcagggcagcccgggcgcgcgcggcccgccgggcctgaaaggcgataaaggcattccgggcgataaaggcgcgaaaggcgaaagcggcctgccggatgtggcgagcctgcgccagcaggtggaagcgctgcagggccaggtgcagcatctgcaggcggcgtttagccagtataaaaaagtggaactgtttccgaacggccagagcgtgggcgaaaaaatttttaaaaccgcgggctttgtgaaaccgtttaccgaagcgcagctgctgtgcacccaggcgggcggccagctggcgagcccgcgcagcgcggcggaaaacgcggcgctgcagcagctggtggtggcgaaaaacgaagcggcgtttctgagcatgaccgatagcaaaaccgaaggcaaatttacctatccgaccggcgaaagcctggtgtatagcaactgggcgccgggcgaaccgaacgatgatggcggcagcagcggcagcgaaaactgcgtggaaatttttaccaacggcaaatggaacgatcgcgcgtgcggcgaaaaacgcctggtggtgtgcgaattt

SEQ ID NO: 20 - DNA, 1074 bp, Artificial Sequence
  Synthetic
gcggaaatgaaaacctatagccatcgcaccaccccgagcgcgtgcaccctggtgatgtgcagcagcgtggaaagcggcctgccgggccgcgatggccgcgatggccgcgaaggcccgcgcggcgaaaaaggcgatccgggcctgccgggcgcggcgggccaggcgggcatgccgggccaggcgggcccggtgggcccgaaaggcgataacggcagcgtgggcgaaccgggcccgaaaggcgataccggcccgagcggcccgccgggcccgccgggcgtgccgggcccggcgggccgcgaaggcccgctgggcaaacagggcaacattggcccgcagggcaaaccgggcccgaaaggcgaagcgggcccgaaaggcgaagtgggcgcgccgggcatgcagggcagcgcgggcgcgcgcggcctggcgggcccgaaaggcgaacgcggcgtgccgggcgaacgcggcgtgccgggcaacaccggcgcggcgggcagcgcgggcgcgatgggcccgcagggcagcccgggcgcgcgcggcccgccgggcctgaaaggcgataaaggcattccgggcgataaaggcgcgaaaggcgaaagcggcctgccggatgtggcgagcctgcgccagcaggtggaagcgctgcagggccaggtgcagcatctgcaggcggcgtttagccagtataaaaaagtggaactgtttccgaacggccagagcgtgggcgaaaaaatttttaaaaccgcgggctttgtgaaaccgtttaccgaagcgcagctgctgtgcacccaggcgggcggccagctggcgagcccgcgcagcgcggcggaaaacgcggcgctgcagcagctggtggtggcgaaaaacgaagcggcgtttctgagcatgaccgatagcaaaaccgaaggcaaatttacctatccgaccggcgaaagcctggtgtatagcaactgggcgccgggcgaaccgaacgatgatggcggcagcagcggcagcgaagattgcgtggaaatttttaccaacggcaaatggaacgatcgcgcgtgcggcgaaaaacgcctggtggtgtgcgaattt

SEQ ID NO: 21 - DNA, 1065 bp, Artificial Sequence
  Synthetic
gcggaaatgaaaacctatagccatcgcaccaccccgagcgcgtgcaccctggtgatgtgcagcagcgtggaaagcggcctgccgggccgcgatggccgcgatggccgcgaaggcccgcgcggcgaaaaaggcgatccgggcctgccgggcgcggcgggccaggcgggcatgccgggccaggcgggcccggtgggcccgaaaggcgataacggcagcgtgggcgaaccgggcccgaaaggcgataccggcccgagcggcccgccgggcccgccgggcgtgccgggcccggcgggccgcgaaggcccgctgggcaaacagggcaacattggcccgcagggcaaaccgggcccgaaaggcgaagcgggcccgaaaggcgaagtgggcgcgccgggcatgcagggcagcgcgggcgcgcgcggcctggcgggcccgaaaggcgaacgcggcgtgccgggcgaacgcggcgtgccgggcaacaccggcgcggcgggcagcgcgggcgcgatgggcccgcagggcagcccgggcgcgcgcggcccgccgggcctgaaaggcgataaaggcattccgggcgataaaggcgcgaaaggcgaaagcggcctgccggatgtggcgagcctgcgccagcaggtggaagcgctgcagggccaggtgcagcatctgcaggcggcgtttagccagtataaaaaagtggaactgtttccgaacggccagagcgtgggcgaaaaaatttttaaaaccgcgggctttgaaaaaccgtttaccgaagcgcagctgctgtgcacccaggcgggcggccagctggcgagcccgcgcagcgcggcggaaaacgcggcgctgcagcagctggtggtggcgcagaacaaagcggcgtttctgagcatgaccgatagcaaaaccgaaggcaaatttacctatccgaccggcgaaagcctggtgtatagcaactgggcgccgggcgaaccgaacgatgatggcggcagcgaagattgcgtggaaatttttaccaacggcaaatggaacgatcgcgcgtgcggcgaaaaacgcctggtggtgtgcgaattt

SEQ ID NO: 22 - DNA, 1065 bp, Artificial Sequence
  Synthetic
gcggaaatgaaaacctatagccatcgcaccaccccgagcgcgtgcaccctggtgatgtgcagcagcgtggaaagcggcctgccgggccgcgatggccgcgatggccgcgaaggcccgcgcggcgaaaaaggcgatccgggcctgccgggcgcggcgggccaggcgggcatgccgggccaggcgggcccggtgggcccgaaaggcgataacggcagcgtgggcgaaccgggcccgaaaggcgataccggcccgagcggcccgccgggcccgccgggcgtgccgggcccggcgggccgcgaaggcccgctgggcaaacagggcaacattggcccgcagggcaaaccgggcccgaaaggcgaagcgggcccgaaaggcgaagtgggcgcgccgggcatgcagggcagcgcgggcgcgcgcggcctggcgggcccgaaaggcgaacgcggcgtgccgggcgaacgcggcgtgccgggcaacaccggcgcggcgggcagcgcgggcgcgatgggcccgcagggcagcccgggcgcgcgcggcccgccgggcctgaaaggcgataaaggcattccgggcgataaaggcgcgaaaggcgaaagcggcctgccggatgtggcgagcctgcgccagcaggtggaagcgctgcagggccaggtgcagcatctgcaggcggcgtttagccagtataaaaaagtggaactgtttccgaacggccagagcgtgggcgaaaaaatttttaaaaccgcgggctttgaaaaaccgtttaccgaagcgcagctgctgtgcacccaggcgggcggccagctggcgagcccgcgcagcgcggcggaaaacgcggcgctgcagcagctggtggtggcgaaaaacgaagcggcgtttctgagcatgaccgatagcaaaaccgaaggcaaatttacctatccgaccggcgaaagcctggtgtatagcaactgggcgccgggcgaaccgaacgatgatggcggcagcgaagattgcgtggaaatttttaccaacggcaaatggaacgatcgcgcgtgcggcgaaaaacgcctggtggtgtgcgaattt

SEQ ID NO: 23 - DNA, 1074 bp, Artificial Sequence
  Synthetic
gcggaaatgaaaacctatagccatcgcaccaccccgagcgcgtgcaccctggtgatgtgcagcagcgtggaaagcggcctgccgggccgcgatggccgcgatggccgcgaaggcccgcgcggcgaaaaaggcgatccgggcctgccgggcgcggcgggccaggcgggcatgccgggccaggcgggcccggtgggcccgaaaggcgataacggcagcgtgggcgaaccgggcccgaaaggcgataccggcccgagcggcccgccgggcccgccgggcgtgccgggcccggcgggccgcgaaggcccgctgggcaaacagggcaacattggcccgcagggcaaaccgggcccgaaaggcgaagcgggcccgaaaggcgaagtgggcgcgccgggcatgcagggcagcgcgggcgcgcgcggcctggcgggcccgaaaggcgaacgcggcgtgccgggcgaacgcggcgtgccgggcaacaccggcgcggcgggcagcgcgggcgcgatgggcccgcagggcagcccgggcgcgcgcggcccgccgggcctgaaaggcgataaaggcattccgggcgataaaggcgcgaaaggcgaaagcggcctgccggatgtggcgagcctgcgccagcaggtggaagcgctgcagggccaggtgcagcatctgcaggcggcgtttagccagtataaaaaagtggaactgtttccgaacggccagagcgtgggcgaaaaaatttttaaaaccgcgggctttgtgaaaccgtttaccgaagcgcagctgctgtgcacccaggcgggcggccagctggcgagcccgcgcagcgcggcggaaaacgcggcgctgcagcagctggtggtggcgaaaaacgaagcggcgtttctgagcatgaccgatagcaaaaccgaaggcaaatttacctatccgaccggcgaaagcctggtgtatagcaactgggcgccgggcgaaccgaacgatgatggcggcggcagcggcagcgaagattgcgtggaaatttttaccaacggcaaatggaacgatcgcgcgtgcggcgaaaaacgcctggtggtgtgcgaattt

SEQ ID NO: 24 - DNA, 1074 bp, Artificial Sequence
  Synthetic
gcggaaatgaaaacctatagccatcgcaccaccccgagcgcgtgcaccctggtgatgtgcagcagcgtggaaagcggcctgccgggccgcgatggccgcgatggccgcgaaggcccgcgcggcgaaaaaggcgatccgggcctgccgggcgcggcgggccaggcgggcatgccgggccaggcgggcccggtgggcccgaaaggcgataacggcagcgtgggcgaaccgggcccgaaaggcgataccggcccgagcggcccgccgggcccgccgggcgtgccgggcccggcgggccgcgaaggcccgctgggcaaacagggcaacattggcccgcagggcaaaccgggcccgaaaggcgaagcgggcccgaaaggcgaagtgggcgcgccgggcatgcagggcagcgcgggcgcgcgcggcctggcgggcccgaaaggcgaacgcggcgtgccgggcgaacgcggcgtgccgggcaacaccggcgcggcgggcagcgcgggcgcgatgggcccgcagggcagcccgggcgcgcgcggcccgccgggcctgaaaggcgataaaggcattccgggcgataaaggcgcgaaaggcgaaagcggcctgccggatgtggcgagcctgcgccagcaggtggaagcgctgcagggccaggtgcagcatctgcaggcggcgtttagccagtataaaaaagtggaactgtttccgaacggccagagcgtgggcgaaaaaatttttaaaaccgcgggctttgtgaaaccgtttaccgaagcgcagctgctgtgcacccaggcgggcggccagctggcgagcccgcgcagcgcggcggaaaacgcggcgctgcagcagctggtggtggcgaaaaacgaagcggcgtttctgagcatgaccgatagcaaaaccgaaggcaaatttacctatccgaccggcgaaagcctggtgtatagcaactgggcgccgggcgaaccgaacgatgatggcggcagcagcggcgcggaagattgcgtggaaatttttaccaacggcaaatggaacgatcgcgcgtgcggcgaaaaacgcctggtggtgtgcgaattt

SEQ ID NO: 25 - DNA, 35 bp, Artificial Sequence
  Synthetic
gcgtctcggatccgcctggagattctgagctctag

SEQ ID NO: 26 - DNA, 29 bp, Artificial Sequence
  Synthetic
gcggccgctgagggaggcgttccataggc

SEQ ID NO: 27 - DNA, 46 bp, Artificial Sequence
  Synthetic
gattttcaagacagcaggctttgaaaaaccatttacggaggcacag

SEQ ID NO: 28 - DNA, 46 bp, Artificial Sequence
  Synthetic
ctgtgcctccgtaaatggtttttcaaagcctgctgtcttgaaaatc

SEQ ID NO: 29 - DNA, 46 bp, Artificial Sequence
  Synthetic
gcaacagctggtcgtagctcagaacaaggctgctttcctgagcatg

SEQ ID NO: 30 - DNA, 46 bp, Artificial Sequence
  Synthetic
catgctcaggaaagcagccttgttctgagctacgaccagctgttgc

SEQ ID NO: 31 - DNA, 44 bp, Artificial Sequence
  Synthetic
caggggagcccaacaatgatggcggcagcagcgggtcagaggac

SEQ ID NO: 32 - DNA, 44 bp, Artificial Sequence
  Synthetic
gtcctctgacccgctgctgccgccatcattgttgggctcccctg

SEQ ID NO: 33 - DNA, 48 bp, Artificial Sequence
  Synthetic
gagcccaacgatgatggcggcagcagcgggtcagaggactgtgtggag

SEQ ID NO: 34 - DNA, 48 bp, Artificial Sequence
  site directed mutagenesis GSS(326a-326b-326)_REV
ctccacacagtcctctgacccgctgctgccgccatcatcgttgggctc

SEQ ID NO: 35 - DNA, 42 bp, Artificial Sequence
  Synthetic
ggcggcagcagcgggtcagagaactgtgtggagatcttcacc

SEQ ID NO: 36 - DNA, 42 bp, Artificial Sequence
  Synthetic
ggtgaagatctccacacagttctctgacccgctgctgccgcc

SEQ ID NO: 37 - DNA, 49 bp, Artificial Sequence
  Synthetic
gcccaacgatgatggcgggtcaagcggagcagaggactgtgtggagatc

SEQ ID NO: 38 - DNA, 49 bp, Artificial Sequence
  Synthetic
gatctccacacagtcctctgctccgcttgacccgccatcatcgttgggc

SEQ ID NO: 39 - DNA, 48 bp, Artificial Sequence
  Synthetic
gagcccaacgatgatggcggcggcagcgggtcagaggactgtgtggag

SEQ ID NO: 40 - DNA, 48 bp, Artificial Sequence
  Synthetic
ctccacacagtcctctgacccgctgccgccgccatcatcgttgggctc

(19) United States
(12) Patent Application Publication
(10) Pub. No.: US 2016/0333074 A1
(43) Pub. Date: Nov. 17, 2016

(54) NOVEL POLYPEPTIDE AND USES THEREOF

(71) Applicant: U-PROTEIN EXPRESS B.V., Houten (NL)

(72) Inventors: Marinus VAN EIJK, Utrecht (NL); Hendrik Peter HAAGSMAN, Zeist (NL)

(73) Assignee: U-PROTEIN EXPRESS B.V., Houten (NL)

(21) Appl. No.: 15/029,887

(22) PCT Filed: Oct. 16, 2014
(86) PCT No.: PCT/NL2014/050720

§ 371 Date: Apr. 15, 2016

(30) Foreign Application Priority Data
Oct. 17, 2013 (NL) 2011626

(51) Int. Cl. C07K 14/785

(52) U.S. Cl. CPC C07K 14/785; A61K 38/00

(57) ABSTRACT

The present invention relates to a novel polypeptide having specific modifications compared to the natural polypeptide. The said novel polypeptide has improved binding characteristics. The present invention also relates to a method of obtaining the said novel polypeptide and to the use of the said novel polypeptides for the treatment of respiratory disorders.

The present invention relates to a polypeptide comprising a modified carbohydrate recognition domain (herein abbreviated as “CRD”) wherein the CRD is derived from human surfactant protein D (herein abbreviated as “hSP-D”). The invention also relates to the use of said modified CRD for the treatment of respiratory diseases, including viral and infectious diseases.

Influenza is a highly contagious respiratory disease, estimated to cause between 250,000 and 500,000 deaths worldwide each year. The disease is caused by infection with influenza A virus (IAV). This virus has the potential to cause pandemic influenza outbreaks in humans as illustrated recently by the swine-origin pandemic flu of 2009. Vaccination of humans is important to generate immune protection against infection and prevention/reduction of disease (severity). However, development and production of effective vaccines is time-consuming and there is general concern that traditional vaccines may not meet the demands of seasonal influenza or pandemic outbreaks of IAV.

In contrast, antiviral drugs can play an important role in providing means of acute protection against viral infections in humans, either as a prophylactic or a therapeutic drug. Currently, two classes of licensed antiviral drugs exist against IAV. Adamantanes (amantadine and rimantadine), which inhibit the proton channel function of the matrix 2 (M2) protein and thereby prevent acidification of the endosomes, needed for correct unfolding of the viral proteins and subsequent fusion with the endosomal membranes that results in release of the viral genes into the cytoplasm. The other class comprises the neuraminidase inhibitors, zanamivir and oseltamivir, substances that reduce the spread of virus by blocking the release of newly formed virions from infected cells.

Unfortunately, currently available antiviral drugs not only show pharmaceutical side effects, but more importantly, the rapid emergence of drug-resistant strains illustrates the limitations of these antivirals. For example, seasonal H3N2 IAV which is resistant to amantadine emerged in 2000 in Asia and has been a clinically relevant strain ever since. Moreover, most of the seasonal H1N1 viruses isolated in the influenza season 2008-2009 were resistant to oseltamivir (Boltz et al., Drugs (2010); 70(11): 1349-1362). Clearly there is a need for novel classes of drugs that can be used in humans to prevent and treat influenza and other important viral infections as effectively as possible.

The present inventors have found that introducing mutations to a naturally occurring protein in humans that is part of the innate immune response to IAV can provide a new class of therapeutic agents, and in particular antiviral agents. The protein on which the new therapeutic agents are based is human surfactant protein D (hSP-D). This protein belongs to a family of proteins known as the ‘collectins’.

Collectins are found in all mammalian species and belong to the C-type lectin superfamily (reviewed by Veldhuizen et al., FEBS J. (2011); 278(20): 3930-3941, included herein by reference). As part of the innate immune system, collectins play a key role in the first line of defence against invading micro-organisms. h-SPD is expressed in respiratory tissues and contributes to the neutralization of a broad spectrum of inhaled microorganisms such as bacteria, fungi and viruses, including IAV (van de Wetering et al., Eur. J. Biochem. (2004); 271(7): 1229-1249).

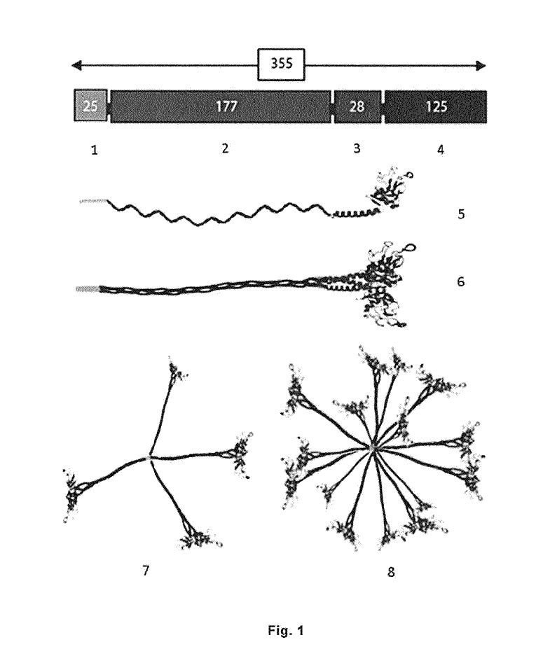

The polypeptide chains of collectins consist of four domains: a cysteine-rich N-terminal domain, a collagen-like domain, an alpha-helical coiled-coil neck domain and a C-terminal carbohydrate-recognition domain (hereinafter also referred to as CRD, and also known in the prior art as ‘lectin domain’; FIG. 1). The polypeptide chains of collectins form trimers that may assemble into larger dodecamers consisting of four trimeric subunits and form a cruciform-like structure. In addition, SP-D can form even higher-order multimers, so-called ‘fuzzy balls’ with a mass of several million Da. (Crouch, E. C., Am. J. Respir. Cell Mol. Biol. (1998); 19(2): 177-201; FIG. 1).

hSP-D interacts with infectious particles via its CRDs that recognize patterns of glycans expressed on the surface of microorganisms. Neutralization is initiated via binding of hSP-D to these pathogens, thereby preventing attachment of pathogens to the delicate pulmonary epithelial surface and reducing the incidence of lung infections. Moreover, this interaction leads to an opsonizing effect which results in enhanced phagocytosis and killing by neutrophils and macrophages. hSP-D also helps to modulate host inflammatory responses in the alveolar space, thereby protecting the lungs from tissue damage that may result from excessive infection-induced inflammation (Wright, J. R., Nat. Rev. Immunol. (2005); 5(1): 58-68).

Collectins interact with glycoconjugates and/or lipid moieties present on the surface of a great variety of micro-organisms and allergens, and with receptors on host cells. Collectins bind a broad range of the unique polysaccharides incorporated in the cell walls of microorganisms. In part, this broad-binding specificity is thought to be achieved by the fact that the CRD's have a very open trough-like binding site, to be found in the so-called shallow groove of the CRD. Further, several collectins, such as hSP-D, can also bind nucleic acids, phospholipids and non-glycosylated proteins (Kuroki et al., Cell Microbiol. (2007); 9(8):1871-1879).

Through these interactions, the collectins play an important role in innate host defence and various defence functions have been reported to date and include a role in agglutination, complement activation, opsonation and activation of phagocytosis, inhibition of microbial growth and modulation of inflammatory responses.

The CRDs of collectins are compactly folded domains of about 115-130 amino acid residues and have been reported to be located at the C-terminus of the collectin protein. Comparison of the CRDs of soluble collectins has revealed that 22 amino acids are conserved within this domain (reviewed by Veldhuizen and co-authors (FEBS J., (2011); 278(20): 3930-3941).

Several molecular structural properties can have an impact on the interactions between collectins and microorganisms: the degree of assembly, oligomeric configuration (e.g. dodecameric vs hexatrimeric), orientation of the CRDs towards the neck domain, sugar binding characteristics of the CRD (specificity and affinity), and length of the collagen domain. In addition, collectins can be post-translationally modified with for example O- and/or N-linked oligosaccharides in the collagen domain and/or in the CRD and these may have implications for pathogen binding. SP-A, for example, has a sialic acid-rich N-glycan in its CRD that interacts with the sialic acid receptor present on the hemagglutinin (HA) of IAV. In contrast to SP-Ds from other species, only SP-D from pigs (pSP-D) also contains such an N-glycan in its CRD. The N-glycan has been shown to contribute to interactions between pSP-D and IAV, in addition to the lectin-mediated interactions between SP-D and oligosaccharide moieties located on the HA of IAV.

M. van Eijk et al describe the binding interactions between the CRD of porcine SP-D (pSP-D) and a viral sugar component. The authors demonstrate that pSP-D exhibits specific structural features in its CRD and it is hypothesized that these porcine-specific structural elements result or contribute to the distinct viral binding properties of pSP-D (van Eijk et al., J. Biol. Chem. (2011); 286(23): 20137-20151).

Given the increase of microbial resistance to therapeutic agents, specifically the rapid increase in viral resistance to antiviral agents, there is an urgent need to develop novel antiviral and antimicrobial agents that are less prone to resistance-development, having high binding affinity to envisaged viruses and microbes, while being tolerated by the host immune system.

In order to meet this need the present inventors have provided a polypeptide comprising a modified carbohydrate recognition domain of human surfactant protein D (hSP-D, SEQ ID NO: 1), the carbohydrate recognition domain encompasses amino acid residues 231-355 of SEQ ID NO: 1, wherein the modification comprises:
(A) a substitution of at least one amino acid residue at a position between amino acid residues 231-355 (inclusive) of SEQ ID NO: 1 by an amino acid belonging to the same physicochemical group as the amino acid at the corresponding position between amino acid residues 231-358 (inclusive) of the carbohydrate recognition domain of porcine surfactant protein D (pSP-D; SEQ ID NO: 2) and/or
(B) insertion between positions 323 and 329 (inclusive) of SEQ ID NO: 1 of at least three contiguous amino acids belonging to the same physicochemical group as amino acid residues at positions 327-329 (inclusive) of SEQ ID NO: 2,
   with the proviso that the modified carbohydrate recognition domain is not identical to the amino acid sequence between positions 245-358 (inclusive) of SEQ ID NO:2.

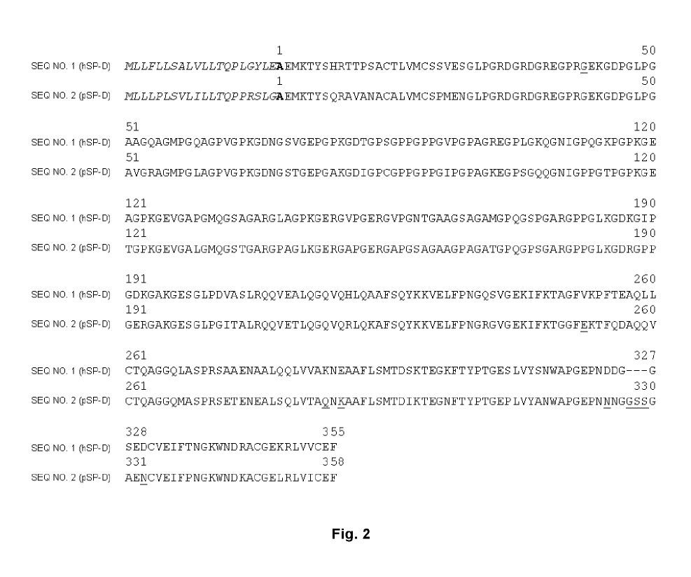

It has been found that the CRD of hSP-D can be modified by one or more amino acid substitutions replacing specific amino acid residues as present in the hSP-D sequence, by amino acid residues that belong to the same physicochemical group (as defined below) as the amino acid residues at a corresponding position in the pSP-D sequence when the sequences of hSP-D and pSP-D are aligned with each other. For example, when SEQ ID NO:1 is aligned with SEQ ID NO: 2, positions 1-326 of SEQ ID NO: 1 correspond to positions 1-326 of SEQ ID NO: 2 and positions 327-355 of SEQ ID NO:1 correspond to positions 330-358 of SEQ ID NO: 2. Amino acids at positions 327-329 in pSP-D are not present in hSP-D (FIG. 2).

It has also been found that the CRD of hSP-D can be modified by insertion of at least three contiguous amino acids which belong to the same physicochemical group (as defined below) as the amino acid residues between 327 and 329 from pSP-D into the amino acid sequence of hSP-D at a position between amino acids 323 and 329 of hSP-D (SEQ ID NO: 1).

The effect of the substitutions and/or insertions in the hSP-D structure is that the antiviral activity of hSP-D is substantially increased relative to the unmodified, natural hSP-D.

The modifications according to the invention correspond to substitutions or insertions in the hSP-D sequence of amino acids belonging to the same physicochemical group as amino acids at the corresponding positions in pSP-D. By physicochemical group is meant a group of amino acids with similar hydrophobicity, charge (positive or negative) or polar properties.

As defined herein, the group of hydrophobic amino acids comprises, for example, alanine (Ala; A), isoleucine (Ile; I), leucine (Leu; L), phenylalanine (Phe; F), valine (Val; V) proline (Pro; P), glycine (Gly; G), methionine (Met; M), tryptophan (Trp; W). These hydrophobic amino acids can further be subdivided into hydrophobic aliphatic amino acids (alanine, glycine, valine, isoleucine, leucine, and methionine) and hydrophobic aromatic amino acids (phenylalanine, tyrosine and tryptophan).

As defined herein, the group of charged amino acids comprises positively or negatively charged amino acids. For example, positively charged amino acids comprise arginine (Arg; R), histidine (His; H) and lysine (Lys; K). For example, negatively charged amino acids comprise aspartic acid (Asp; D) and glutamic acid (Glu; E).

As defined herein, the group of polar amino acids comprises, for example, serine (Ser; S), threonine (Thr; T), asparagine (Asn; N), glutamine (Glu; Q), tyrosine (Tyr; Y), and cysteine (Cys; C).

The aforementioned hydrophobic, charged (positive or negative) or polar amino acids belong to the group of (human) proteinogenic amino acids. However, the hydrophobic, charged (positive or negative) or polar may be chosen from the group comprising non-proteinogenic (that is, amino acids not encoded by triplet codons in humans) amino acids for example from β-amino acids (β3 and β2), homo-amino acids, or D-amino acids, however, α-amino acids are preferred.

The inventors have found that by substituting an amino acid in hSP-D by an amino acid with the same or similar physicochemical properties as the amino acid at the corresponding position in pSP-D leads to a polypeptide with improved enhanced binding to carbohydrates and therefore improved activity, for example antiviral activity. For example hSP-D has a valine at position 251. pSP-D has glutamic acid at the corresponding position. Glutamic acid is one of the negatively charged amino acids according to the above classification. According to the invention, this means that the valine on position 251 of hSP-D may be replaced by any negatively charged amino acid of the same physicochemical group, i.e. not only by glutamic acid (which is the amino acid in pSP-D on that position) but also by aspartic acid or for example by a non-proteinogenic amino acid. Examples of non-proteinogenic amino acids of glutamic acid include, for example carboxyglutamic acid or for example D-glutamic acid.

It has therefore been surprisingly shown that an amino acid in hSP-D may be replaced by an amino acid with dissimilar physicochemical properties, i.e. belonging to another physicochemical group as defined above. In other words a hydrophobic amino acid in hSP-D can be replaced by any negatively charged amino acid as such a negatively charged amino acid is present in pSP-D on the said position. This is surprising as mutations involving a change in physicochemical properties at a particular position in a protein often leads to destabilised polypeptides, and therefore ultimately leads to a decrease in activity of the polypeptide. Replacing e.g. hydrophobic valine at position 251 in natural hSP-D by e.g. negatively charged glutamic acid, instead of having a detrimental effect on hSP-D, actually improves the antiviral activity of hSP-D.

Furthermore it has been found that insertion of at least three contiguous amino acids between positions 323 and 329 in the CRD of hSP-D can lead to an increase in the binding carbohydrates to the novel polypeptide according to the invention.

According to the invention, the at least three contiguous amino acids corresponds not only to the glycine-serine-serine motif that is present in the pSP-D at position 327-329 but also to three contiguous amino acids that belong to the same physicochemical group as the glycine-serine-serine motif.

Analogous to the discussion above, by ‘belonging to the same physicochemical group’ is meant amino acids which have the same physicochemical properties as the corresponding amino acids in pSP-D. In other words, the insertion can be summarized as being a motif composed of a sequence of a hydrophobic amino acid followed by two polar amino acids, i.e. hydrophobic-polar-polar amino acids respectively. Glycine belongs to the group of hydrophobic amino acids and can, for example, be replaced by another hydrophobic amino acid, for example alanine. Serine belongs to the group of polar amino acids and thus can be replaced by other polar amino acids, for example by threonine.

Without wishing to be bound by theory, it is understood that the insertion of at least three contiguous amino acids with hydrophobic, polar, and polar properties, respectively, can improve the binding of hSP-D to a carbohydrate or oligosaccharide. The glycine-serine-serine motif in pSP-D forms a loop on the surface of the protein and the inventors have found that when the hydrophobic-polar-polar amino acid motif is inserted in the CRD of hSP-D between positions 323 and 329, a loop is also formed. It is thought that the loop provides potential for increased hydrogen bonds and van der Waal's contacts between, for example, an oligomannose substrate and hSP-D. The presence of extra binding interactions between the modified CRD and a viral carbohydrate or oligosaccharide may therefore explain the observed increased (antiviral) activity of the polypeptide according to the invention.

By “at least three contiguous amino acids” is meant that the insertion may comprise additional amino acids as defined elsewhere in this application, for example a fourth amino acid or indeed a fifth amino acid. The additional amino acids may be any amino acid but preferentially those amino acids that contribute to extension or stabilisation of the loop structures near the ligand binding site, or amino acids that provide potential for hydrogen bonding to the carbohydrate in the carbohydrate binding site, as defined elsewhere in this application.

Without wishing to be bound by theory it is understood that the above mentioned modifications, that is substitution or insertion, introduced into the CRD of hSP-D improve the binding of the CRD to an invading IAV or other microbe. Both pSP-D and hSP-D bind carbohydrates, for example N-linked glycans present on the surface of IAV. By modifying the sequence of the CRD of hSP-D by substitution or insertion of amino acids belonging to the same physicochemical group as amino acids at the corresponding position in pSP-D, it is surprisingly found that interactions between the modified CRD of hSP-D and the viral carbohydrates increases relative to the natural (unmodified) CRD of hSP-D. This improved binding interaction therefore enhances the ability of the modified CRD of hSP-D to bind IAV and prevent infection of pulmonary tissues by IAV and other microbes more efficiently than natural hSP-D. In addition, this mutant shows enhanced inhibitory activity against a broader range of IAV strains and, possibly, other microorganisms.

To the knowledge of the inventors there has been no disclosure in the art of polypeptides comprising a mutant CRD based on the combination of CRD sequences from hSP-D and pSP-D, which polypeptides have improved activity towards, for example, their role in the (innate) immune system or as part of an anti-infective composition to combat infections in humans.

The term “polypeptide” refers to a chain of covalently attached amino acids that are joined by peptide bonds. Polypeptide chains typically fold into a compact, stable and functional form that is referred to as a protein. Proteins can assemble into higher order structures which can be composed of one or more equal or different polypeptides. When the term “polypeptide” is used, within the context of the current invention, also proteins and/or chimeras comprising such polypeptide are to be understood, or truncated derivatives such as neck-CRD trimeric fragments, either with or without the presence of a small part of the collagen domain preceding the neck domain as described for example by Hakansson, K., et al. (Structure (1999); 7(3): 255-64) and by van Eijk et al (J. Biol. Chem. (2012); 287(32): 26666-26677).

The term “natural polypeptide” refers to the amino acid sequence of a polypeptide as found in nature. Such a polypeptide is also referred to in the art as “wild-type” or “native” polypeptide.

The term “carbohydrate recognition domain” which is also referred to herein as CRD, is known in the art and in this document, refers to the lectin domain as part of a collectin molecule. The CRD of collectins is a C-type lectin domain and this makes collectins a member of the C-type lectin domain family. This domain, in the presence of calcium ions, is capable of binding carbohydrates. The clustering of three CRDs enables multivalent binding and therefore recognition of specific patterns of oligosaccharides that, for example, are found on the surface of a wide variety of micro-organisms. (Crouch, E. C., Am. J. Respir. Cell Mol. Biol. (1998); 19(2) 177-201; Holmskov, U., et al. Immunol. Today (1994):'15(2): 67-74; Thiel et al., FEBS Lett. (1989); 250(1): 78-84).

The term “modified CRD” denotes a CRD that has an altered molecular structure in comparison to the (natural) CRD it is derived from. In particular, “modified CRD” denotes the presence of one or more substitutions/insertions/deletions of specific amino acid residues in the amino acid sequence resulting in the “modified CRD” in comparison to the (natural) CRD it is derived from.

The term “amino acid substitution” means the replacement of one or more amino acid residues in the amino acid sequence of the naturally occurring (natural) human SP-D (SEQ NO: 1) by a proteinogenic amino acid or a non-proteinogenic amino acid belonging to the same physicochemical group as an amino acid from the corresponding position in pSP-D (SEQ ID NO:2).

The term “corresponding position” refers to the positions of amino acids when two or more sequences are aligned with each other. For example, when SEQ ID NO:1 is aligned with SEQ ID NO: 2, positions 1-326 of SEQ ID NO: 2 correspond to positions 1-326 of SEQ ID NO: 2 and positions 327-355 of SEQ ID NO:1 correspond to positions 330-358 of SEQ ID NO: 2.

The term “with the proviso that the modified carbohydrate recognition domain is not identical to the amino acid sequence between positions 245-358 (inclusive) of SEQ ID NO:2 means the amino acid sequence of modified hSP-D comprises at least one amino acid that is different to the amino acid at the corresponding position in the amino acid sequence between positions 245-358 (inclusive) of pSP-D (SEQ ID NO:2).

The inventors have identified a number of modifications that once introduced in the CRD of hSP-D, results in a mutant hSP-D that shows improved antimicrobial and antiviral activity.

In an embodiment there is provided a polypeptide wherein the substitution comprises at least one amino acid chosen from the group consisting of V251E, K287Q, E289K, D324N and D330N, or a combination of two or more thereof.

Surprisingly it has been found that introducing substitutions to the CRD of hSP-D at one or more of the positions described above, the antiviral activity of hSP-D against IAV or another microbe can be improved. For example, mutating the valine at position 251 to glutamic acid in the amino acid sequence of hSP-D can further improve the said activity of hSP-D. Also, mutating the lysine at position 287 to glutamine in the amino acid sequence of hSP-D can further improve the said activity of hSP-D. In addition, mutating the glutamic acid at position 289 to a lysine can further improve the said activity of hSP-D. Moreover, mutating the aspartic acid at position 324 to an asparagine can further improve the said activity of hSP-D. Additionally, mutating the aspartic acid at position 330 to an asparagine can further improve the said activity of hSP-D.

As described above the substitution may be a single point mutation, for example valine, at position 251. In doing so it has been surprisingly shown that non-conservative mutations lead to an SP-D with increased activity. By non-conservative mutations is meant that an amino acid in the natural polypeptide is substituted by an amino acid which has dissimilar physicochemical properties in terms of hydrophobicity, charge or basicity. This is surprising as non-conservative mutations often lead to destabilised polypeptides, and therefore a decrease in activity of the polypeptide. Replacing hydrophobic valine at position 251 in natural hSP-D by polar glutamic acid, instead of having a detrimental effect on hSP-D, actually improves the activity of hSP-D with regard to its antiviral activity.

In an embodiment there is provided a polypeptide, wherein the insertion comprises 3 to 8 amino acids, preferably 3 to 7 amino acids, more preferably 3 to 6 amino acids, even more preferably 3 to 5 amino acids, still even more preferably 3 to 4 amino acids, most preferably 3 amino acids

The naturally occurring hSP-D sequence does not comprise the three contiguous amino acids present at positions 327-329 in pSP-D. In pSP-D the three amino acids at positions 327-329 form a loop motif. The effect of the said insertion into hSP-D is that a loop motif is formed that protrudes from the surface of hSP-D so that more binding interactions can occur between the carbohydrate and the hSP-D. Surprisingly it has been found that an insert of at least three contiguous amino acids provides sufficient improved binding to the carbohydrate in the carbohydrate binding site to improve, for example, the antiviral activity of the hSP-D relative to the unmodified hSP-D.

The insertion of at least three contiguous amino acids into hSP-D can be part of an insertion of up to eight amino acids. In this embodiment, up to eight amino acids can be inserted wherefrom three of the eight inserted amino acids correspond to amino acids which belong to the same physicochemical group as the amino acids present at positions 327-329 of pSP-D. Although eight amino acids may be inserted, it has been found that preferably seven amino acids, more preferably six amino acids, even more preferably five amino acids, still more preferably four amino acids, most preferably three amino acids are inserted. An insertion of more than eight amino acids may cause steric effects that disrupt the binding of hSP-D to a microbe and therefore an insertion of more than eight amino acids is not preferred.

In an embodiment there is provided a polypeptide wherein the insertion is between positions 324 and 328 (inclusive), preferably 325 and 327 (inclusive), most preferably between positions 326 and 327 (inclusive) of SEQ ID NO: 1.

The naturally occurring hSP-D does not comprise the three amino acids present at positions 327-329 in pSP-D. In pSP-D the amino acids present at positions 327-329 form a loop. By inserting three contiguous amino acids at a position between 324 and 328 of hSP-D, a loop is also formed on the hSP-D surface, at a site close to the carbohydrate binding site. The close proximity of the inserted loop to the carbohydrate binding site may increase the favourable interactions between hSP-D and the carbohydrate, which ultimately leads to an increased (antiviral) activity.

As discussed above at least three contiguous amino acids can take place at a single position between two amino acids in the sequence of hSP-D, for example between positions 324 and 325, preferably between 325 and 326, more preferably between 327 and 328, even more preferably between 326 and 327. For example, if the insertion takes place between positions 326 and 327 of hSP-D, the inserted amino acids are designated as 326a, 326b and 326c. Insertion between 326 and 327 is particularly preferred as this position leads to high binding to, for example, the microbial carbohydrate.

In an embodiment there is provided a polypeptide wherein the insertion comprises at least one glycine.

Preferably in an embodiment, when the inserted three contiguous amino acids comprise at least one glycine residue the modified hSP-D is able to bind carbohydrates with higher affinity. Without wishing to be bound by theory, it is proposed that the glycine residue increases the flexibility of the loop. The effect of the flexible loop is that such a loop is able to move in order to accommodate different carbohydrates.

In an embodiment there is provided a polypeptide wherein the insertion comprises at least one serine, preferably at least two serines.

Preferably in an embodiment, when the inserted amino acids comprise at least one serine the modified hSP-D is able to bind carbohydrates with higher affinity. Without wishing to be bound by theory, the hydroxyl group of the serine side chain may form hydrogen bonds with the carbohydrate in the binding site, increasing the stability of the hSP-D-carbohydrate complex. Preferably, two serines are present in order to provide even more hydrogen bonds with the carbohydrate in the binding site.

In an embodiment there is provided a polypeptide wherein the modification comprises insertion of the contiguous amino acid residues Gly-Ser-Ser (G-S-S).

In another preferred embodiment, when the at least three contiguous amino acids corresponds to Gly-Ser-Ser as present at positions 327-329 of pSP-D, improved binding of the hSP-D to carbohydrates, on for example a microbe, is observed relative to unmodified hSP-D is observed.

Without wishing to be bound by theory, it is understood that the insertion of the described motif, in particular Gly-Ser-Ser motif can improve the binding of hSP-D to a carbohydrate or oligosaccharide. The Gly-Ser-Ser motif forms a flexible loop, as shown in molecular docking and crystallographic studies (M. van Eijk et al, supra), that has the potential for increased hydrogen bonds and van der Waal's contacts between, for example, an oligomannose substrate and pSP-D. The presence of potential extra binding interactions between the modified CRD and a viral carbohydrate or oligosaccharide may therefore explain the observed increased (antiviral) activity of the polypeptide according to the invention.

In an embodiment there is provided a polypeptide wherein the modification comprises an insertion of the contiguous amino acids Gly-Ser-Ser (G-S-S) corresponding to amino acid residues at positions 327-329 of SEQ ID NO:2 between positions 326 and 327 of SEQ ID NO: 1.

Although insertion of the Gly-Ser-Ser motif is also possible at other positions within the CRD sequence of hSP-D as described above, these amino acids are preferably inserted at the position where these amino acids are found in the pSP-D sequence when aligned with hSP-D, i.e. between positions 326 and 327 in hSP-D. In this embodiment, the entire ‘GSS-loop’ present in pSP-D is therefore incorporated at the corresponding location in the hSP-D sequence (between position 326 and 327 of SEQ ID NO: 1). It can be seen in FIG. 2 that hSP-D does not contain the Gly-Ser-Ser motif between amino acid 326 and 327. It is therefore surprising that an insertion of the Gly-Ser-Ser at the position stated is tolerated by the protein and does not have a detrimental effect on the structure of hSP-D but in fact results in an improvement in the activity of hSP-D.

It has been advantageously found that insertion of the amino acid sequence Gly-Ser-Ser in the amino acid sequence of the CRD of hSP-D between positions 326 and 327 in hSP-D can further improve the activity of the polypeptide, for example in binding to IAV.

It will be clear from the above for a person skilled in the art that a number of modifications to the sequence of the CRD of hSP-D are possible in order to improve the activity of hSP-D, for example the antiviral activity. The modifications may be amino acid substitutions and/or insertions as described above. The inventors have found in a preferred embodiment a polypeptide wherein the modification comprises amino acid substitutions V251E, K287Q, E289K, D324N and D330N, and insertions 326aG, 326bS and 326cS.

The inventors have found that when the CRD of hSP-D is modified by carrying out amino acid substitutions V251E, K287Q, E289K, D324N and D330N, in combination with inserting the Gly-Ser-Ser motif between positions 326 and 327 of hSP-D, a polypeptide is obtained that has a particular high activity, for example as an antiviral agent against different strains of IAV.

Without wishing to be bound by theory, it is understood that substitution or insertion of amino acids at the aforementioned eight positions in hSP-D improves the binding of hSP-D to the microbial surface. The binding is thought to be improved by maximising the potential extra binding interactions between the modified CRD of hSP-D and a microbial carbohydrate or oligosaccharide. Consequently the antimicrobial activity of hSP-D is increased.

Importantly, not all the amino acids which are dissimilar when the CRDs of hSP-D and pSP-D are compared, need to be mutated in order to dramatically increase the activity of hSP-D. Alignment of hSP-D with pSP-D shows that the following positions vary: V251E, P253T, T255Q, E256D, L259Q, L260V, 1268M, A274E, A275T, A278E, Q281S, V285T, K287Q, E289K, S2981, K303N, 5311P, S315A, D324N, D325N, 326aG, 326bS, 326cS, S328A, D330N, T336P, R343K, K348L and V3521. Furthermore, three contiguous amino acids, present in pSP-D at positions 327, 328 and 329 of the pSP-D sequence but absent in natural hSP-D, are indicated in the hSP-D sequence as (virtual) positions 326a, b and c, respectively, in order to allow a proper alignment of the corresponding positions between both pSP-D and hSP-D.

The inventors have shown that a limited number of mutations, for example eight mutations, improve the antiviral activity of the resulting hSP-D as compared to the natural hSP-D. This has the advantage that the novel antiviral agent has a high degree of identity with ‘natural’ hSP-D and is therefore considered an attractive drug as the modified hSP-D is less likely to generate unwanted side effects in humans (e.g. immunogenic, immunotoxic) than xenoproteins such as pSP-D.

It can be envisaged that a number of different modifications are possible, for example the polypeptide according to the invention preferably comprises at least two amino acid modifications, preferably the polypeptide according to the invention comprises at least three amino acid modifications, preferably the polypeptide according to the invention comprises at least four amino acid modifications, preferably the polypeptide according to the invention comprises at least five amino acid modifications, preferably the polypeptide according to the invention comprises at least six amino acid modifications, preferably the polypeptide according to the invention comprises at least seven amino acid modifications, preferably the polypeptide according to the invention comprises at least eight amino acid modifications.

In another embodiment, the polypeptide according to the invention further comprises a substitution of one or more amino acids at the positions, chosen from 325, 335 and 343 of SEQ ID NO: 1 by another amino acid, preferably chosen from the group, consisting of glycine (Gly; G), alanine (Ala; A), valine (Val; V), leucine (Leu; L), isoleucine (Ile; I), methionine (Met; M), phenylalanine (Phe, F) tryptophan (Trp; W), serine (Ser; S), threonine (Thr; T), cysteine (Cys; C), tyrosine (Tyr; Y), asparagine (Asn, N), glutamine (Gln; Q), aspartic acid (Asp, D), glutamic acid (Glu; E), lysine (Lys; K), proline (Pro; P), histidine (His; H) and arginine (Arg; R). Polypeptide according to any one of the previous claims, wherein the modification further comprises a substitution of one or more amino acids at the positions chosen from 325, 335 and 343 of SEQ ID NO: 1 by another amino acid, preferably chosen from the group, consisting of glycine (Gly; G), alanine (Ala; A), valine (Val; V), leucine (Leu; L), isoleucine (Ile; I), methionine (Met; M), phenylalanine (Phe: F) tryptophan (Trp; W), serine (Ser; S), threonine (Thr; T), cysteine (Cys; C), tyrosine (Tyr; Y), asparagine (Asn; N), glutamine (Gin; Q), aspartic acid (Asp; D), glutamic acid (Glu; E), lysine (Lys; K), proline (Pro: P), histidine (His; H) and arginine (Arg; R), more preferably the amino acid at position 325 is alanine, more preferably the amino acid at position 335 is tyrosine or more preferably amino acid at position 343 is valine.

In a preferred embodiment, aspartic acid at position 325 in hSP-D is preferably replaced by alanine In another preferred embodiment phenylalanine at position 335 in hSP-D is replaced by tyrosine. In a further preferred embodiment arginine at position 343 in hSP-D is preferably replaced by valine. It is particularly preferred that aspartic acid, pheylalanine and tyrosine present at positions 325, 335 and 343 respectively are replaced with alanine, tyrosine and valine respectively. The modified hSPD therefore comprises three substitutions at positions 325, 335 and 343 of hSP-D.

The term ‘another amino acid’ is intended to mean that the amino acid at the said position 325, 335 and 343 is different from aspartic acid on position 325, from phenylalanine on position 335 and from arginine on position 343, as these amino acids are present on their respective positions in the hSP-D sequence. Furthermore, ‘another amino acid’ is understood to include both proteinogenic and non-proteinogenic amino acids.

Without wishing to be bound by theory, in hSP-D-carbohydrate complexes, occupation of the carbohydrate binding site by a suitable carbohydrate moiety allows additional neighbouring carbohydrate moieties to participate in secondary stabilizing interactions with residues flanking the carbohydrate binding site. Such ‘extended site’ interactions between carbohydrates and side chains of Asp325, Phe335 or Arg343, of the hSP-D CRD appear to contribute to observed species differences in ligand recognition by SP-D. A polypeptide according to the invention further comprising at least one modification in the amino acids responsible for the extended site interactions, can lead to an increased activity of the said polypeptide.

In a further embodiment, there is provided a polypeptide according to the invention, wherein the modification further comprises introduction of at least one glycosylation site in the said CRD.

The term “glycosylation site” is known in the art and refers to a site within a polypeptide which is susceptible to forming a covalent bond with a carbohydrate, or carbohydrate-chain, for example by post-translational modification processes occurring in eukaryotes or archaea, or by (bio) chemical treatment in the presence of enzymes. Collectins, and in particular the binding properties of the CRD's, have been subject to various types of research and have been proposed for various uses. For example, WO 2007/111496 describes introduction of N-glycosylation sites in the CRD of hSP-D to increase the anti-IAV activity of hSP-D compared to natural hSP-D that does not contain an N-glycosylation site in its CRD.

Without being bound by any theory, it is believed that by the introduction of at least one N-glycosylation site within the CRD of hSP-D, a polypeptide is provided that comprises at least one introduced glycosylation site which is positioned within the CRD such that binding to, for example, micro-organisms is improved.

In a further embodiment, there is provided a polypeptide according to the invention, wherein the introduced glycosylation site has the amino acid sequence Asn-X-Ser or Asn-X-Thr, wherein X can be any amino acid except proline.

Within the preferred glycosylation sites of the invention, X can be any amino acid except proline. For example, X can be chosen from the group consisting of (between brackets 3 letter code followed by 1 letter code) glycine (Gly; G), alanine (Ala; A), valine (Val; V), leucine (Leu; L), isoleucine (Ile; I), methionine (Met; M), phenylalanine (Phe; F), tryptophan (Trp; W), serine (Ser; S), threonine (Thr; T), cysteine (Cys; C), tyrosine (Tyr; Y), asparagine (Asn; N), glutamine (Gln; Q), aspartic acid (Asp; D), glutamic acid (Glu; E), lysine (Lys; K), arginine (Arg; R), and histidine (His; H). It was found that the preferred glycosylation sites according to the invention are those glycosylation sites that are susceptible to N-linked glycosylation, i.e. wherein glycosylation (i.e. insertion of saccharides) occurs to the amide nitrogen of an asparagine side chain.

The term “N-linked glycosylation” is known in the art. In brief, N-linked glycosylation occurs in eukaryotes and widely in archaea, but rarely in prokaryotes, although prokaryotes might also be capable of glycosylation after substitution or insertion of enzymes required for glycosylation into these prokaryotes. In typical cases, for N-linked oligosaccharides, a saccharide-precursor is first added to the asparagine in a polypeptide chain of a protein. The structure of this precursor can for example consist of 3 glucose, 9 mannose, and 2 N-acetyl glucosamine molecules. There are three major types of N-linked saccharides: high-mannose oligosaccharides, complex oligosaccharides, and hybrid forms of both. High-mannose relates to in essence two N-acetylglucosamines with many mannose residues, whereas complex oligosaccharides can contain almost any number of other types of monosaccharides, and can include the original N-acetylglucosamines.

Proteins can be glycosylated by these types of saccharides and in different domains of the protein. During glycosylation, residues can be cleaved off, be added or be modified (for example, elongated with a variety of different monosaccharides including galactose, N-acetylglucosamine, N-acetylgalactosamine, fucose and sialic acid).

In a further embodiment, there is provided a polypeptide according to the invention, wherein the modification further comprises introduction of at least two glycosylation sites in the said CRD.

Surprisingly, it has been found that the presence of at least two glycosylation sites in the CRD may further improve the use and/or activity of the polypeptide. For example, the presence of at least two glycosylation sites in the CRD increases binding to different and more types, species and the like of different micro-organisms, for example, different strains of IAVs. It will be clear for a person in the art that depending on the desired properties of the polypeptide, one, two, three, four, five, or more glycosylation sites can be introduced in the CRD.

In a preferred embodiment, there is provided a polypeptide according to the invention, wherein said introduced glycosylation site is glycosylated with a carbohydrate. Such carbohydrate can be any carbohydrate/saccharide, as discussed above. The polypeptide can be glycosylated by means of an enzyme, for example present in a organism wherein the polypeptide is expressed, for example human embryonic kidney 293E cells, or for example by treatment of a purified polypeptide or protein in the presence of enzymes required for glycosylation, as discussed above, or by any chemical means. It has been found that glycosylation of the introduced glycosylation site in the CRD of the polypeptide is required for improving the use and/or activity of the polypeptide, for example for increased binding of the polypeptide to particular micro-organisms, for example pathogens, for example influenza A viruses. As will be appreciated by the person skilled in the art, depending on the desired properties of the proteins, for example with respect to the microorganisms whereto the polypeptide or protein should bind, or with respect to the desired strength of binding (as will be exemplified in the examples below), it might be required to glycosylate the introduced glycosylation site with the same, or different types of carbohydrates.

In a preferred embodiment the said carbohydrate, that is or will be used for glycosylating the introduced glycosylation site in the CRD, comprises at least one sialic acid residue, preferably at least one terminal sialic acid residue.

It has been found that the presence of sialic acid residues on the carbohydrate that is attached to the polypeptide at the introduced glycosylation site of the collectin CRD, can dramatically improve the use and/or activity of the polypeptide.

For example, glycosylation of a newly introduced glycosylation site in the CRD of hSP-D, and subsequent glycosylation/sialylation, can dramatically improve binding of the polypeptide, for example as part of a mature protein, to various influenza A viruses.

Without being bound by theory, it is believed that this improved activity is related to binding of the sialic acid residue to, for example, the sialic acid receptor (s) present on a micro-organism, for example present on or within a hemagglutinin, a viral spike glycoprotein present on the surface of virus particles.

In another embodiment, there is provided a polypeptide according to the invention, wherein said sialic acid residue is linked to the carbohydrate by alpha (2, 3)-linkage or alpha (2, 6)-linkage, or a mixture thereof.

It has been found that a sialic acid that is linked to the carbohydrate, more particular to a penultimate galactose residue, by either alpha (2, 3) or alpha (2, 6)-linkage, is in particular favourable in a polypeptide according to the invention.

It has been found that depending on the desired use of the polypeptide, for example for binding to IAV isolated from human, avian, equine or porcine species, sialic acids linked to the carbohydrate by alpha (2, 3)-linkage or alpha (2, 6)-linkage might be preferred.

For example, human IAVs appear to preferentially bind to an alpha (2, 6)-linked sialic acids, those isolated from birds and equines appears preferentially to alpha (2, 3)-linked sialic acids, while porcine IAVs tend to recognise sialic acids that are linked either alpha (2, 3) or alpha (2, 6) to the penultimate saccharide of complex oligosaccharides present (or artificially introduced as described above) in the polypeptides.

From the above, the polypeptide according to the invention may also comprise a combination of amino acid modifications derived from pSP-D. The polypeptide may therefore comprise a mutation (substitution or insertion) of at least one amino acid derived from pSP-D and insertion of at least one glycosylation site, preferably derived from the sequence of pSP-D.

In a preferred embodiment, there is provided a polypeptide wherein said introduced glycosylation site is introduced at a position between amino acid 240 and 260, preferably between 246 and 253, and/or between 267 and 298, preferably between 272 and 290, and/or between 304 and 331, preferably between 331 and 344, in the amino acid sequence of said CRD.

It has been found that the substitution or insertion of at least one amino acid mutation derived from pSP-D at the sites indicated in hSP-D, provides a polypeptide which shows improved binding to various micro-organisms like IAV and, as indicated above, can be used in the treatment of infections by such micro-organisms.

Without wishing to be bound by theory, it is believed that the substitution or insertion of at least one modification as described above might be favourably situated in the exterior of the CRD which results in, for example, well exposed N-linked oligosaccharides or, for example in the interior of the ligand binding pocket of CRD whereby CRD-ligand bonding interactions are improved. The favourable positions of the amino acid modifications thereby improve the use and/or activity of hSP-D within the context of the invention.

In another embodiment, there is provided a polypeptide according to the invention, wherein the polypeptide comprises an N-terminal domain comprising cysteine residues and/or a collagen-like domain characterised by repetitive Gly-Xaa-Yaa sequences and/or a neck-domain.

The terms “collagen-like domain characterised by repetitive Gly-Xaa-Yaa” and “neck-domain” are known to a person skilled in the art, and are for example reviewed by van de Wetering (2004). Typically, a “collagen-like domain characterised by repetitive triplet Gly-Xaa-Yaa sequences” refers to a domain which can make three polypeptides trimerise into a collagen triple-helix (resulting in one collagen trimeric subunit) as, for example, has been shown for hSP-D (Persson-A et al., Biochemistry 1989, 28(15): 6361-6367) and surfactant protein A (Haagsman-H P et al, Am J Physiol. 1989, 257 (6Pt1): L421-429).

The process of trimerisation is suggested to be triggered by the neck region of collectins (Hoppe, H. J., P. N. Barlow, and K. B. M. Reid. “A parallel three stranded alpha-helical bundle at the nucleation site of collagen triple-helix formation.” FEBS Lett. 344.2-3 (1994): 191-195).

Typically, a neck-domain is a short alpha-helical coiled-coil domain which initiates trimerisation of three monomers due to a heptad repeat of hydrophobic residues, resulting in strong hydrophobic interactions between the polypeptide chains in this domain.

It has been found that a polypeptide according to the invention, wherein the polypeptide comprises an N-terminal region comprising cysteine residues and/or a collagen-like domain characterised by repetitive Gly-Xaa-Yaa sequences and/or a neck-domain can show improved use and/or activity within the context of the invention.

In yet another embodiment there is provided a polypeptide according to any one of the previous claims wherein said polypeptide is in the form of a multimer, preferably a trimer, preferably hexamer, preferably nonamer, more preferably a dodecamer, even more preferably a multidodecamer.

It is believed that the formation of a multimer, for example a dodecamer, might enable improved target recognition (for example virus recognition), but also allows simultaneous and multivalent interactions with the target, thereby improving efficient binding to said target. The dodecamer is a four armed structure in which each arm represents a trimer of the polypeptide according to the invention. Multimers, comprising polypeptides according to the invention can show improved binding to various micro-organisms, in particular to IAVs. Such polypeptides according to the invention can therefore suitably be used in for example the treatment of infections with such micro-organisms, or inflammation that results from the infection.

The person skilled in the art knows properties that are required within a polypeptide to allow for multimerisation, and can for example comprise the presence of a collagen-like region characterised by repetitive triplet Gly-Xaa-Yaa sequences (wherein Xaa and Yaa can be any amino acid), as described above.

The polypeptides that multimerise can be identical or different, for example with respect to the presence of introduced glycosylation sites in the CRD. For example, a trimer can be formed which comprises 1, 2 or 3 identical polypeptides

In an embodiment of the present invention, there is provided a polypeptide according to one of the previous claims, wherein said polypeptide is a non-natural polypeptide.

As will be understood by the person skilled in the art, multimers, preferably trimers, comprising at least one non-natural polypeptide, are also within the scope of the invention. The term “non-natural polypeptide” relates to a polypeptide that does not normally occur in nature. For example, such polypeptide does not normally occur in more than 70% of individual animals of a particular species (including human beings), i.e. does not occur in 7 out of 10 randomly selected individual animals of a particular species, and, obviously, wherein said non-natural polypeptide according to the invention is introduced in the animal by for example recombinant techniques. A non-natural polypeptide is also understood to comprise non-natural amino acids as described above.

It has been found that there can be provided non-natural polypeptides according to the invention with improved use and/or activity, in comparison to the natural occurring polypeptides, within the context of the invention.

The polypeptides according to the invention have broad applicability, for example in research, and various suitable methods for manipulating, expressing, isolating, modifying, assaying, and the like are readily available (Molecular Cloning: A Laboratory Manual Second Edition. Sambrook, J., Fritsch, E. and Maniatis, T. Cold Spring Harbor Press, Cold Spring Harbor, N.Y.; Handbook of Molecular and Cellular Methods in Biology and Medicine, ed. Kaufman, P., Wu, W. and Kim, D. CRC Press, Boca Raton, Fla. (1995)).

DNA, RNA and/or cDNA sequences for hSP-D and pSP-D are publically available from the NCBI database (www.ncbi.nlm.nih.gov), for example for human SP-D: gene bank accession number X65018; and for pSP-D: gene bank accession number AF132496.

In an embodiment of the present invention, there is provided a polypeptide for use in the treatment of a disease or the prophylactic treatment of an animal body, preferably a mammalian body, more preferably a human body.

The polypeptides according to the invention can suitably be used in the treatment of an animal body, preferably a human body, and can show improved activity in the treatment of, for example, infections with various micro-organisms, for example pathogens, in particular with viruses, more particular influenza A or influenza B viruses.

As will be understood by a person skilled in the art, within the context of the invention treatment also includes prevention of the occurrence of a disease or inhibiting the development of a disease, for example as a consequence of an infection, for example caused by IAV. Additionally, polypeptides according to the invention can be provided, that can be useful in the treatment and/or prevention of bird flu and the like. In other words, the polypeptide according to the present invention can be used as a prophylactic drug. In other words, “treatment” refers to both therapeutic treatment and prophylactic or preventative measures. Those in need of treatment include those already with a disorder, as well as those in which the disorder is to be prevented.

In an embodiment of the present invention, there is provided the use of a polypeptide according to the invention in the treatment respiratory diseases.

It has been found that the polypeptide according to the invention is particular useful in treating respiratory disease. By respiratory diseases is meant pathological conditions affecting the organs and tissues that make gas exchange possible in higher organisms, and includes conditions of the upper respiratory tract, trachea, bronchi, bronchioles, alveoli, pleura and pleural cavity, and the nerves and muscles of breathing.

In an embodiment of the present invention, there is provided the use of a polypeptide according to the invention in the treatment of respiratory diseases, wherein the respiratory disease is chosen from the group consisting of viral infections, bacterial infections, fungal infections and inflammatory disorders.

The inventors have found that the polypeptides according to the invention are effective against a range of infective agents, for example viruses, bacteria and fungi. It has also been found that the polypeptides according to the invention are also active against inflammatory disorders. Without wishing to be bound by theory, the enhanced activity of the modified CRD of hSP-D according to the invention is due to altered binding characteristics of the modified hSP-D to the surface of pathogens and host cells.

In an embodiment of the present invention, there is provided the use of a polypeptide according to the invention wherein the viral infection to be treated is chosen from the group consisting of the influenza A virus, influenza B virus, or influenza C virus, Ebola virus, rhinoviruses, coronaviruses, parainfluenza viruses, adenoviruses, herpesviruses, respiratory syncytial virus, metapneumovirus and enteroviruses vaccinia virus.

The inventors have found that the polypeptides according to the invention are particularly effective against a range of infective viral agents possibly binding to the carbohydrate present on the viral capsid.

In an embodiment of the present invention, there is provided the use of a polypeptide according to the invention wherein the viral infection to be treated is chosen from the group consisting of the genera influenza virus A, influenza B virus and influenza C virus, wherein the genus influenza virus A comprises subtypes H1N1, H2N2, H3N2, H5N1, H7N7, H1N2, H9N2, H7N2H7N3, H10N7, H7N9.

It has been shown that a polypeptide according to the invention can be useful in the treatment of an infection, in particular a genera of the orthomyxoviridae family, for example influenza A virus, influenza B virus and influenza C virus. A polypeptide according to the invention can, for example, be highly effective in the treatment of a specific subtype of IAV, preferably H1N1 or even more preferably H3N2.

In an embodiment of the present invention, there is provided the use of a polypeptide according to the invention wherein the bacterial infection to be treated is chosen from the group consisting of Bacillus anthracis; Bordetella pertussis; Borrelia burgdorferi; Brucella abortus; Brucella canis; Brucella melitensis; Brucella suis; Campylobacter jejuni; Chlamydia pneumonia; Chlamydia trachomatis; Chlamydophila psittaci; Clostridium botulinum; Clostridium difficile; Clostridium perfringens Corynebacterium diphtheria; Enterococcus faecalis and Enterococcus faecium; Escherichia coli sp. in particular, Enterotoxigenic Escherichia coli (ETEC), Enteropathogenic E. coli, E. coli O157:H7; Francisella tularensis; Helicobacter pylori; Haemophilus influenza; Klebsiella pneumonia; Legionella pneumophila; Leptospira interrogans; Listeria monocytogenes; Mycobacterium leprae; Mycobacterium tuberculosis; Neisseria gonorrhoeae; Neisseria meningitides; Pseudomonas aeruginosa; Salmonella enteritidis; Salmonella typhimurium; Shigella sonnei; and Staphylococcus aureus; Streptococcus pneumoniae.

The inventors have found that in a preferred embodiment the modified CRD of hSP-D according to the invention is able to act as an anti-infective agent against bacteria. Without wishing to be bound by theory, the improved activity of the modified hSP-D relative to the natural hSP-D is due to the better binding interaction between the carbohydrates on the surface of the bacteria and the CRD of the hSP-D. The improved binding interaction is a direct result of the inventive substitutions and insertions described above.

In an embodiment, there is provided the use of a polypeptide according to the invention wherein the fungal disease to be treated is chosen from the group consisting of aspergillosis; blastomycosis; candidiasis; coccidioidomycosis; cryptococcosis; histoplasmosis; mucormycosis; pneumocystis; pneumonia, and sporotrichosis.

The inventors have found in a preferred embodiment that the modified CRD of hSP-D according to the invention is able to act as an anti-infective agent against a range of fungi. Without wishing to be bound by theory, the improved activity of the modified hSP-D relative to the natural hSP-D is due to the better binding interaction between the carbohydrates on the surface of fungi and the CRD of the hSP-D. The improved binding interaction is a direct result of the inventive substitutions and insertions described above.

In an embodiment, there is provided the use of a polypeptide according to the invention wherein the inflammatory disorder to be treated is chosen from the group consisting of asthma, autoimmune neutropenia, acute respiratory distress syndrome (ARDS), bronchopulmonary dysplasia (BPD), chronic obstructive pulmonary disorder (COPD), cystic fibrosis, emphysema, hay fever, and sinusitis.

It has also been found that polypeptides according to the invention have an improved activity regarding immune modulation compared to natural hSP-D. Without wishing to be bound by theory, the improved activity of the modified CRD according to the invention enables hSP-D to bind better to lipopolysaccharide and thereby reduce binding of lipopolysaccharide to receptors that trigger immune responses.

In an embodiment, there is provided the use of a polypeptide according to the invention wherein the polypeptide is present as an additive in surfactant formulations that are being used in, for example, treatment of premature new-borns. The surfactant formulation may contain an animal derived surfactant. In some embodiments, the animal derived surfactant is a commercially available surfactant, such as ALVEOFACT®, CUROSURF®, INFASURF®, or SURVANTA®. In some embodiments, the animal derived surfactant is BLES®, SURFACEN®, or CLSE®. In some embodiments, the surfactant formulation contains a synthetic surfactant. In some embodiments, the synthetic surfactant is a commercially available synthetic surfactant, such as EXOSURF®, PUMACTANT®, SURFAXIN®, AEROSURF®, VENTICUTE®, or CHF 5633. In some embodiments, a combination treatment of the polypeptide according to the invention and an animal surfactant is provided. In some of these embodiments, the animal surfactant contains at least one surfactant protein and at least one lipid. For example, in some embodiments, the surfactant formulation contains dipalmitoylphosphatidylcholine (DPPC). In some embodiments, the formulation contains DPPC and at least one of phosphatidylglycerol (PG) and phosphatidylinositol (PI).

In an embodiment, there is provided the use of a polypeptide according to the invention wherein the disease to be treated is chosen from the group consisting of arthritis, eczema, Coeliac disease, Crohn's disease, psoriasis, septic shock syndrome, toxic shock syndrome.

It has been found that polypeptides according to the invention are also valuable agents to reduce the symptoms of allergic conditions, for example arthritis, eczema, coeliac disease, Crohn's disease, psoriasis, septic shock syndrome, toxic shock syndrome.

Furthermore, a polypeptide according to the invention can be used in the treatment of chronic infectious diseases for example, human immunodeficiency virus (HIV), diseases related with congenital or acquired immunodeficiency (AIDS), tuberculosis (T.B) and Hepatitis B.

Other uses and/or activities involving a role for the polypeptides according to the invention include, but are not limited to, carbohydrate binding or interactions with proteins present in or on a micro-organism, improving agglutination, complement activation, opsonisation and activation of phagocytosis, inhibition of microbial growth and modulation of inflammatory responses.

In an embodiment of the present invention, there is provided the use of a polypeptide according to the invention for use in the treatment of influenza disease in combination with an antiviral drug, for example, the antiviral agent is chosen from the group consisting of neuraminidase inhibitors and adamantanes, wherein the neuraminidase inhibitor is chosen from the group consisting of Laninamivir, Oseltamivir, Zanamivir and Peramivir and the adamantane is chosen from the group consisting of rimantadine and adamantine.

It has been found that a polypeptide according to the invention can be used in combination treatment strategy whereby a polypeptide according to the invention is combined with a currently used anti-viral agent. The advantage of this strategy is that the polypeptide according to the invention targets a different aspect of the virus compared to for example neuraminidase inhibitors. Without wishing to be bound by theory, a polypeptide according to the invention prevents virus from binding to the epithelial surface whereas a neuraminidase inhibitor blocks the function of the viral neuraminidase protein thus preventing release of virus and spread of infection. A polypeptide according to the invention can therefore prevent binding of a virus, whereas a neuraminidase inhibitor is only active after the virus has entered a host cell and helps to block release of newly formed virions from the infected host cell, thereby limiting spread of infection.

In an embodiment of the present invention, there is provided the use of a polypeptide according to the present invention for the manufacturing of a medicament for the treatment of respiratory diseases.

Such medicament can further comprise usual compounds like stabilisers, preservatives, pharmaceutical adjuvants and the like. The medicament can be in any suitable administration form like a spray, a fluid, a powder and the like.

In an embodiment of the present invention, there is provided a nucleic acid comprising a sequence of nucleotides encoding a polypeptide according to the invention.

It has been found that such nucleic acid can favourably be used to produce large quantities of a polypeptide according to the invention. Such nucleic acid can be expressed in a well-known prokaryotic or eukaryotic expression system, such as bacteria, yeast or human cell lines.

In an embodiment of the present invention, there is provided a method for obtaining a polypeptide according to the present invention wherein said method comprises the step of introducing an amino acid mutation in the said CRD.

As above, suitable methods are known to the person skilled in the art, and include, for example, site-directed mutagenesis, recombinant-DNA-techniques and the like as described in: Molecular Cloning: A Laboratory Manual Second Edition. Sambrook, J., Fritsch, E. and Maniatis, T. Cold Spring Harbor Press, Cold Spring Harbor, N.Y.; Handbook of Molecular and Cellular Methods in Biology and Medicine, ed. Kaufman, P., Wu, W. and Kim, D. CRC Press, Boca Raton, Fla. (1995).

Accordingly, said substitution or insertion comprises substitution, deletion and/or insertion of at least one amino acid residue in said CRD of hSP-D. As above, suitable methods are known to the person skilled in the art, and include, for example, DNA-cloning procedures, site-directed mutagenesis, recombinant DNA expression techniques and the like.

Suitable hosts can be determined by straight-forward experimentation, and include, for example, prokaryotic and eukaryotic hosts like bacteria, yeast cells and human cells.

As described above, the amino acid with the same physicochemical properties as a corresponding amino acid from pSP-D may be a non-proteinogenic amino acid. The non-proteinogenic amino acid can be introduced by chemical or molecular biology techniques that are known to the person skilled in the art. To effect the substitution or insertion of a modification of a non-proteinogenic amino acid, for example acetyl-lysine, by chemical means, the use of protein semi-synthesis can be considered, as reviewed by Simon, P et al. Org. Biomol. Chem., 10. (2012): 56842-5697, or by molecular biology techniques as summarized by Hendrickson, T. L. Annual Rev. Biochem., 73, (2004): 147-176.

In an embodiment of the present invention, there is provided a method for obtaining a polypeptide according to the present invention wherein said method comprises the step of introducing a glycosylation site in the said CRD.

Suitable methods are known to the person skilled in the art, and include, for example, site-directed mutagenesis, recombinant-DNA-techniques and the like.

In an embodiment of the present invention, there is provided a method for obtaining a polypeptide according to the present invention wherein said method comprises the steps of a) expressing a nucleic acid encoding a polypeptide according to the invention in a host organism under conditions that the said polypeptide is formed and b) isolating the said polypeptide.

The skilled person can determine suitable hosts for the expression of nucleic acid according to the invention by straight-forward experimentation, and include, for example, prokaryotic and eukaryotic hosts like bacteria, yeast cells and human cells.

In an embodiment where a glycosylation site is present, preferably but not necessarily, the host is capable of glycosylating and/or sialylating the introduced glycosylation site in the polypeptide according to the invention, for example human embryonic kidney 293E cells. Methods for isolating the formed polypeptide according to the invention are known to the person skilled in the art, and can include, for example, affinity chromatography, (SDS-page) gel electrophoresis and the like, for example as described by van Eijk et al. (van Eijk, M., et al. “Porcine surfactant protein D is N-glycosylated in its CRD and is assembled into differently charged oligomers.” Am. J. Respir. Cell Mol. Biol. 26.6 (2002): 739-47).

As will be understood by the person skilled in the art, isolating refers to any relative enrichment of the polypeptide according to the invention in comparison to a stadium prior to isolating, for example by increasing the percentage of the polypeptide relative to other proteins present in a solution, or concentrating the polypeptide according to the invention.

Expressing a nucleic acid encoding a polypeptide, according to the invention, in a cell capable of N-glycosylation of proteins and/or capable of sialylation, can advantageously be applied to provide for a polypeptide according to the invention, or a multimer according to the invention, that is glycosylated with a suitable sialic acid-containing carbohydrate at the introduced glycosylation site, thus rendering it unnecessary to glycosylate and/or sialylate by other (bio) chemical means.

The invention will be illustrated by the following figures.

FIG. 1: The molecular structure of surfactant protein D (SP-D).

FIG. 2: Alignment of the amino acid sequence of human SP-D (hSP-D, SEQ. ID NO: 1) and porcine SP-D (pSP-D, SEQ. ID NO: 0.2).

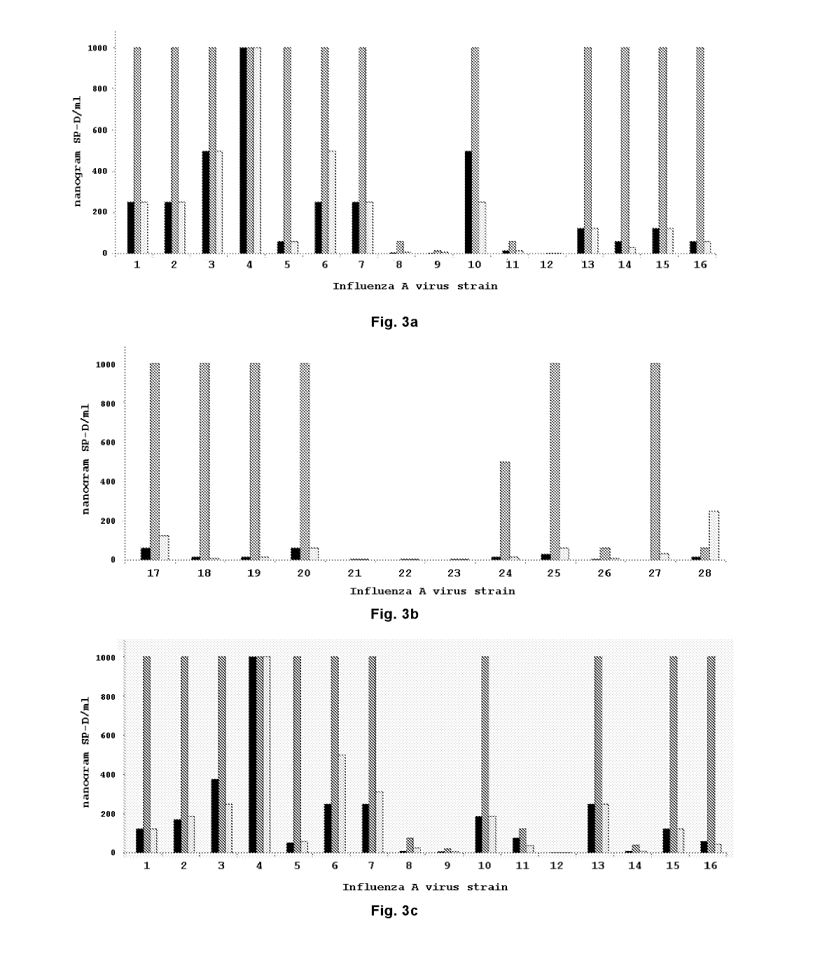

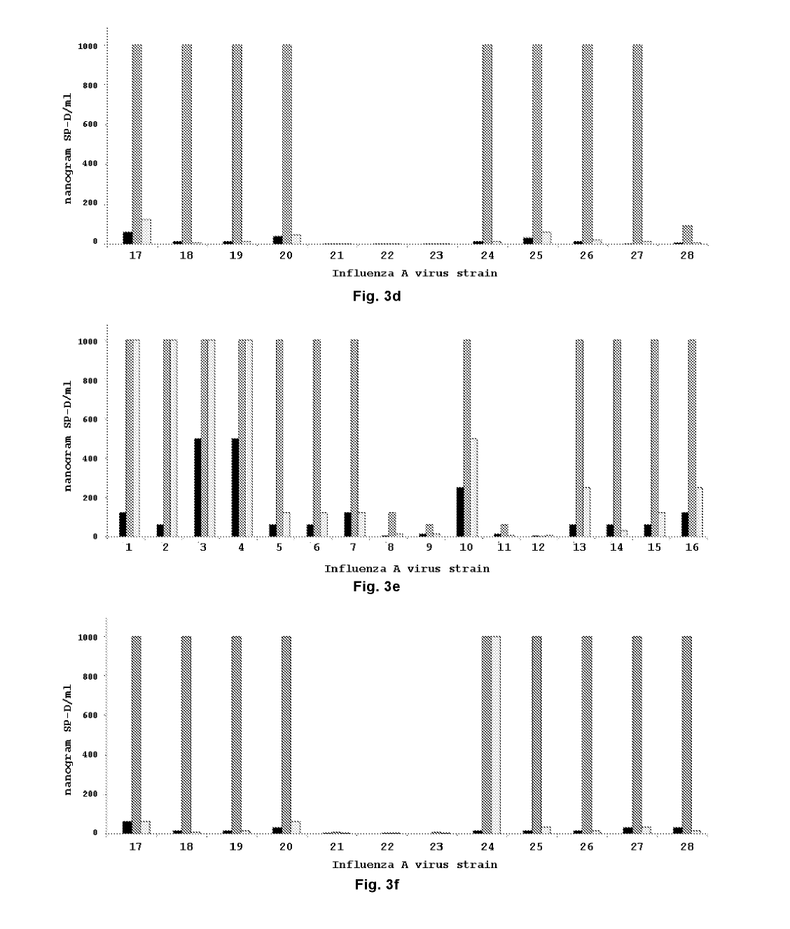

FIG. 3a-f: Comparison of the IAV hemagglutination inhibition activity by the polypeptide according to the invention and other SP-Ds.

### DETAILED DESCRIPTION OF FIGURES

FIG. 1: the molecular structure of surfactant protein D (SP-D). Schematic domain organization of the polypeptide chain of SP-D, consisting of a cysteine-rich N-terminal domain (1), a collagen-like domain (2), a short domain that initiates trimerisation by forming a short alpha-helical coiled-coil between three polypeptide chains (3), and a C-type lectin domain, also referred to as CRD (4), that recognizes glycan structures in a Ca²⁺-dependent manner. The number of amino acids that comprises a mature human SP-D polypeptide and its distinct domains are indicated, and represented in cartoon form as monomer 5. Trimers (6), resulting in well-defined spatial arrangements of three CRDs, are important for glycan pattern-mediated recognition of non-self structures (e.g. pathogens). Trimeric subunits can be further assembled into higher-order oligomers (dodecamers (7) or multidodecamers (8)).

FIG. 2: Alignment of the amino acid sequence of human SP-D (hSP-D, SEQ. ID NO: 1) and porcine SP-D (pSP-D, SEQ. ID NO.2). Amino acid residue numbers of the mature polypeptide are indicated for both hSP-D and pSP-D. The leader peptide is shown in italics. Dashes indicate spaces that are inserted to maximize the identity across the alignment. Underbars show the pSP-D residues that have been transferred to the hSP-D sequence to generate the hSP-D mutant described in the experimental results of this patent application.

FIG. 3a-f: Comparison of the IAV hemagglutination inhibition activity between the polypeptide according to the invention and other SP-Ds. Hemagglutination inhibition data comparing the inhibitory activity of recombinant SP-D derived from pig (natural sequence of porcine SP-D, black bars); from human (natural sequence of human SP-D, grey bars); and a mutant based upon the same human SP-D sequence having 8 porcine-specific residues present in the CRD of human SP-D as described in the present invention (white bars). Graphs represent the amount of SP-D (ng/ml) needed to prevent the virus-induced agglutination of erythrocytes. The differences in inhibitory activity between the three SP-D's are shown for a total of 28 different influenza A virus strains that represent two subtypes. All strains with subtype H1N1 are displayed in FIGS. 3a, 3c and 3e; all strains that belong to subtype H3N2 are displayed in FIGS. 3b, 3d and 3f. Strains were isolated from 3 different host species (swine, human and avian). Strain numbers (X-axis) refer to the numbers in Table 1 to provide details of the individual strains used. The experiments were performed on three different days of analysis, using different batches of protein, and were executed each day in duplicate. Data obtained from each day of analysis are summarized as follows:

FIG. 3a: H1N1 day 1

FIG. 3b: H3N2 day 1

FIG. 3c: H1N1 day 2

FIG. 3d: H3N2 day 2

FIG. 3e: H1N1 day 3

FIG. 3f: H3N2 day 3

The current invention will be further exemplified by the examples below. It is to be understood that the given examples do not impose any particular limitation with respect to embodiments of the invention.

### EXAMPLES

### Example 1

### Preparation of Construct for Full-Length pSP-D and its Expression and Purification

Total RNA, extracted from adult porcine lung tissue, was used in the iScript cDNA synthesis reaction following the manufacturer's recommendations to obtain porcine lung cDNA. This was used as a template in a standard 50-μl PCR using proofstart DNA polymerase to amplify the full coding sequence of pSP-D including the signal peptide sequence, starting 8 nucleotides downstream the start of transcription ending 142 nucleotides downstream from the stop codon (total length: 1315 bp). For subcloning purposes, the forward primer included a BsmBI/BamHI restriction site (PSPDFOR, gcgtctcggatcc GCC TGG AGA TTC TGA GCT CTA G, SEQ ID NO:25). All DNA sequences described in this study are 5′ to 3′ with gene-specific sequences in capital letters) and the reverse primer contained a NotI sequence (PSPDREV, 5′-gcggccgc TGA GGG AGG CGT TCC ATA GGC-3′ SEQ ID NO: 26). The amplified DNA fragment was gel purified and 3′ A-overhangs were added by incubating 10 min at 72° C. in the presence of 1 unit of Taq DNA polymerase and 0.4 mm dATP. The gel purified product was cloned into pCR4-TOPO sequencing vector using a TOPO T/A cloning kit (Invitrogen). Insert-positive clones were isolated and sequenced to rule out any errors. The full-length pSP-D construct was digested with BsmBI/NotI, gel extracted, and ligated into pUPE-101-01 expression vector, a modified version of the pTT3 expression vector. No tags were used. Subclones containing the cDNA were identified by BamHI/NotI restriction analysis. After amplification in TOP10 E. Coli, purified expression clones were transfected into HEK293-EBNA cells. After five days, medium was harvested and used for purification of secreted recombinant pSP-D by mannan affinity chromatography (calcium-dependent binding) and gel filtration (size exclusion) according to van Eijk (van Eijk et al., “Porcine surfactant protein D is N-glycosylated in its CRD and is assembled into differently charged oligomers.”, Am. J. Respir. Cell Mol. Biol. 26.6 (2002): 739-47).

### Example 2

### Preparation of Construct for Full-Length hSP-D and its Expression and Purification

RhSP-D was produced using the full-length hSP-D cDNA clone provided by Dr E. C. Crouch (Washington University, St. Louis, Mo.). This clone was used as a template for PCR cloning into pCR4-TOPO as described for RpSP-D in the previous section, to introduce BsmBI/BamHI (5′-end) and NotI (3′-end) restriction sites to accommodate subcloning in pUPE-101.01. The construct used contained the signal sequence of hSP-D. Procedures for subcloning and recombinant expression and purification of recombinant hSP-D were performed as described in example 1 for pSP-D.

### Example 3

### Site-Directed Mutagenesis on Full-Length hSP-D

Site-directed mutagenesis was performed on the full-length hSP-D cDNA clone in pCR4-TOPO vector (produced as described in example 2) by overlap extension PCR using the QuikChange II site-directed mutagenesis kit according to the manufacturer's instructions (Stratagene, La Jolla, Calif. 92037, USA). Custom-made forward and reverse primers were used to introduce the desired modifications by PCR (see below). Entire sequence and desired modifications were checked by DNA sequencing followed by cloning into pUPE 101.01 expression vectors. Procedures for subcloning and recombinant expression and purification of recombinant hSP-D mutants were performed as described in example 1 for recombinant pSP-D.

Primers used to introduce modifications into the human SP-D sequence via site-directed mutagenesis (change human SP-D residues into porcine-specific SP-D residues).

Underlined: the desired mutation in primer sequence. The pSP-D-sequence-specific amino acids that were substituted or inserted in the hSP-D sequence are also underlined in the amino acid sequence alignment of FIG. 2.

V251E
Forward:
(SEQ ID NO: 27)
5′-GATTTTCAAGACAGCAGGCTTTG
AAAAACCATTTACGGAGGCACAG-3′
Reverse:
(SEQ ID NO: 28)
5′-CTGTGCCTCCGTAAATGGTTTTT
CAAAGCCTGCTGTCTTGAAAATC-3′
K287Q and E289K
Forward:
(SEQ ID NO: 29)
5′-GCAACAGCTGGTCGTAGCTCAGA
ACAAGGCTGCTTTCCTGAGCATG-3′
Reverse:
(SEQ ID NO: 30)
5′-CATGCTCAGGAAAGCAGCCTTGT
TCTGAGCTACGACCAGCTGTTGC-3′
D324N
Forward:
(SEQ ID NO: 31)
5′-CAGGGGAGCCCAACAATGATGGC
GGCAGCAGCGGGTCAGAGGAC-3′
(Italics: GSS loop included in primer design;
1st round of mutation was GSS insertion).
Reverse:
(SEQ ID NO: 32)
5′-GTCCTCTGACCCGCTGCTGCCGC
CATCATTGTTGGGCTCCCCTG-3′
GSS326-LOOP (insertion)
Forward:
(SEQ ID NO: 33)
5′-GAGCCCAACGATGATGGCGGCAGC
AGCGGGTCAGAGGACTGTGTGGAG-3′
Reverse:
(SEQ ID NO: 34)
5′-CTCCACACAGTCCTCTGACCCGCT
GCTGCCGCCATCATCGTTGGGCTC-3′
D330N
Forward:
(SEQ ID NO: 35)
5′-GGCGGCAGCAGCGGGTCAGAGAAC
TGTGTGGAGATCTTCACC-3′
(Italics: GSS loop included in primer design;
1st round of mutation was GSS insertion).
Reverse:
(SEQ ID NO: 36)
5′-GGTGAAGATCTCCACACAGTTCTC
TGACCCGCTGCTGCCGCC-3′
SGA (328a-328b-328c)
Forward:
(SEQ ID NO: 37)
5′-GCCCAACGATGATGGCGGGTCAAGC
GGAGCAGAGGACTGTGTGGAGATC-3′
Reverse:
(SEQ ID NO: 38)
5′-GATCTCCACACAGTCCTCTGCTCCG
CTTGACCCGCCATCATCGTTGGGC-3′
GGS(326a-326b-326c)
Forward:
(SEQ ID NO: 39)
5′-GAGCCCAACGATGATGGCGGCGGCA
GCGGGTCAGAGGACTGTGTGGAG-3′
Reverse:
(SEQ ID NO: 40)
5′-CTCCACACAGTCCTCTGACCCGCTG
CCGCCGCCATCATCGTTGGGCTC-3′

The resulting polypeptides (with corresponding sequence identities) encoded by the thus modified vectors are:
GSS (326a-326b-326c)+V251E+K287Q+E289K+D324N+D330N (SEQ ID NO: 3) which comprises a loop insertion and five substitutions as indicated compared to the protein of SEQ ID NO: 1.
GSS (326a-326b-326c)+V251E+K287Q+E289K+D324N (SEQ ID NO: 4) which comprises a loop insertion and four substitutions as indicated compared to the protein of SEQ ID NO: 1.
GSS (326a-326b-326c)+V251E+K287Q+E289K (SEQ ID NO. 5) which comprises a loop insertion and three substitutions as indicated compared to the protein of SEQ ID NO: 1.
GSS (326a-326b-326c)+D330N+D324N (SEQ ID NO: 6) which comprises a loop insertion and two substitutions as indicated compared to the protein of SEQ ID NO:1.
GSS (326a-326b-326c)+D330N (SEQ ID NO: 7) which comprises a loop insertion and one substitution as indicated compared to the protein of

SEQ ID NO:1.
GSS (326a-326b-326c) (SEQ ID NO: 8) which comprises the a loop insertion as indicated compared to the protein of SEQ ID NO:1.
V251E+K287Q+E289K (SEQ ID NO: 9) which comprises the three substitutions as indicated compared to the protein of SEQ ID NO:1.
V251E (SEQ ID NO. 10) which comprises one substitution as indicated compared to the protein of SEQ ID NO:1.
GGS (326a-326b-326c) (SEQ ID NO. 11) which comprises a loop insertion as indicated compared to the protein of SEQ ID NO:1.
SGA (328a-328b-328c) (SEQ ID NO. 12): which comprises a loop insertion as indicated compared to the protein of SEQ ID NO:1.

### Example 4

### Influenza a Virus Preparations

Twenty eight viruses were selected based on their subtype and species of origin and included avian swine and human IAV of the H1N1 and H3N2 subtypes. The characteristics of these strains are listed in Table 1. All the viruses were propagated in Madin-Darby Canine Kidney (MDCK, ATCC CCL-34) cells as described by Rimmelzwaan et al. (Virol Methods (1998) 74: 57-66). Briefly, the viruses were passaged in MDCK cells once and subsequently biologically cloned by passaging in MDCK cells three times under limiting dilution conditions. Finally, a virus stock was produced by collecting culture supernatants of the infected MDCK, clarified by low speed centrifugation, and aliquoted and stored at −135° C. in the presence of 25% saccharose until use. The infectious virus titers (TCID50/ml) of the stocks were assessed by titering MDCK cells.

TABLE 1
Influenza A virus strains used in this study

| # | Details strain |
|---|---|
| 1 | A/swine/Ned/602/09(H1N1) |
| 2 | A/swine/Cal/4/09(H1N1) |
| 3 | A/swine/Shope/11/56(H1N1) |
| 4 | A/swine/Iowa/15/30(H1N1) |
| 5 | A/swine/New Jersey/8/76(H1N1) |
| 6 | A/swine/Ned/25/80(H1N1) |
| 7 | A/swine/Ned/1/87(H1N1) |
| 8 | A/Ned/364/06(H1N1) |
| 9 | A/Ned/246(H1N1) |
| 10 | A/Puerto Rico/8/34(H1N1) |
| 11 | A/Ned/26/07(H1N1) |
| 12 | A/USSR/90/77(H1N1) |
| 13 | A/mallard/Ned/15/05(H1N1) |
| 14 | A/mallard/Ned/26/06(H1N1) |
| 15 | A/whitefronted Goose/Ned/1/07(H1N1) |
| 16 | A/commonTeal/Ned/10/00(H1N1) |
| 17 | A/swine/Oedenrode/7C/96(H3N2) |
| 18 | A/swine/Ned/849/93(H3N2) |
| 19 | A/swine/Utrecht/4/85(H3N2) |
| 20 | A/swine/Ukkel/1/84(H3N2) |
| 21 | A/Ned/348/07(H3N2) |
| 22 | A/Ned/548/05(H3N2) |
| 23 | A/Ned/312/03(H3N2) |
| 24 | A/Ned/35/93(H3N2) |
| 25 | A/mallard/Ned/19/05(H3N2) |
| 26 | A/mallard/Ned/51/08(H3N2) |
| 27 | A/mallard/Swe/57/03(H3N2) |
| 28 | A/mallard/Ned/1/07(H3N2) |

### Example 5

### Hemagglutination Inhibition Assay

Binding of SP-D to the viral hemagglutinin and interference with binding of the virus to its receptor was assessed by a hemagglutination inhibition (HI) assay. Two-fold dilutions of SP-D or peanut agglutinin (Sigma Aldrich, Schneeldorf, Germany), which was included as a negative control, were made using Dulbecco's phosphate buffered saline containing 1 mM of CaCl₂ and 0.5 mM of MgCl₂, (PBS-CM; Gibco, Grand Island, USA). To 100 μl of the diluted SP-D, 2 hemagglutination units (HAU) of the respective viruses diluted in PBS-CM were added. After 1 h, 25 μl of 1% turkey erythrocytes were added. The hemagglutination patterns were read after 3 h of incubation at room temperature. As a negative control, the experiment was also performed in PBS without CaCl₂ to demonstrate the Ca²⁺-dependency of the SP-D activity. Results of the HI measurements to compare the HI-activity of recombinant wild-type pSP-D (SEQ ID NO:2), recombinant wild-type hSP-D (SEQ ID NO:1), and the recombinant hSP-D-mutant (SEQ ID NO: 3) against a total of 28 different IAV strains are listed in FIG. 3a-f.

An indicated value of 1000 ng/ml indicates that the amount of SP-D needed to prevent agglutination by a specific influenza A virus strain is at least 1000 ng/ml or higher (the strain being resistant against the highest dose of SP-D tested in this assay which was 500 ng/ml).

It is clear from the results that the polypeptide according to the invention has improved activity, for example with respect to inhibition of the hemagglutinating activity of most IAV strains that have been tested (H1N1 and H3N2 subtypes of IAV). The polypeptide according to the invention provides new and surprising means for the use of modified hSP-D in the treatment of IAV.

### Example 6

The hSP-D mutant proteins SEQ ID NO:3-SEQ ID NO:12 were screened for activity against strain 10 (A/Puerto Rico/8/34(H1N1)) of the IAV strains tested in Example 5 The results can be seen in Table 2.

TABLE 2
Hemagglutination inhibition activity of hSP-D
mutants against IAV (A/Puerto Rico/8/34(H1N1)

| | | IAV inhibitory |
|---|---|---|
| | | activity relative to |
| | | hSP-D (wild type, |
| Mutant | SEQ ID NO:# | SEQ ID NO: 1) |
| GSS(326a-326b-326c) + | SEQ ID NO: 3 | +++++ |
| V251E + K287Q + E289K + | | |
| D324N + D330N | | |
| GSS(326a-326b-326c) + | SEQ ID NO: 4 | ++++ |
| V251E + K287Q + E289K + | | |
| D324N | | |
| GSS(326a-326b-326c) + | SEQ ID NO. 5 | ++++ |
| V251E + K287Q + E289K | | |
| GSS(326a-326b-326c) + D330N + | SEQ ID NO: 6 | ++++ |
| D324N | | |
| GSS(326a-326b-326c) + D330N | SEQ ID NO: 7 | +++ |
| GSS(326a-326b-326c) | SEQ ID NO: 8 | +++ |
| V251E + K287Q + E289K | SEQ ID NO: 9 | +++ |
| V251E | SEQ ID NO. 10 | + |
| GGS(326a-326b-326c) | SEQ ID NO. 11 | − |
| (comparative example) | | |
| SGA(328a-328b-328c) | SEQ ID NO. 12 | −− |
| (comparative example) | | |

Key:
−− not active;
− same as control
+ low activity;
+++ = moderate activity;
+++++ high activity

It is clear from the results shown in Table 2 that the polypeptides according to the invention have improved inhibitory activity relative to wild type hSP-D (SEQ ID NO:1).

hSP-D mutants according to the invention having a loop insertion (for example, SEQ ID NO:8), show improved activity relative to wild type hSP-D. Similarly, substitution of at least one amino acid residue (for example, SEQ ID NO:9) in the sequence of hSP-D (SEQ ID NO:1) is also sufficient to result in enhanced IAV inhibitory activity. hSP-D mutants according to the invention possessing both a loop insertion and substitution of, at least one amino acid residue for example SEQ ID NO:7), preferably more than one amino acid residue for example SEQ ID NO: 3 and SEQ ID NO: 5), also lead to increased inhibitory activity against IAV, relative to wild type hSP-D (SEQ ID NO:1).

1-35. (canceled)

36. Polypeptide comprising a modified carbohydrate recognition domain of human surfactant protein D (hSP-D, SEQ ID NO 1), the carbohydrate recognition domain encompasses amino acid residues 231-355 of SEQ ID NO: 1, wherein the modification comprises:
(A) a substitution of at least one amino acid residue at a position between amino acid residues 231-355 (inclusive) of SEQ ID NO: 1 by an amino acid belonging to the same physicochemical group as the amino acid at the corresponding position between amino acid residues 231-358 (inclusive) of the carbohydrate recognition domain of porcine surfactant protein D (pSP-D; SEQ ID NO: 2) and/or
(B) insertion between positions 323 and 329 (inclusive) of SEQ ID NO: 1 of at least three contiguous amino acids belonging to the same physicochemical group as amino acid residues at positions 327-329 (inclusive) of SEQ ID NO: 2,
with the proviso that the modified carbohydrate recognition domain is not identical to the amino acid sequence between positions 245-358 (inclusive) of SEQ ID NO: 2.

37. Polypeptide according to claim 36, wherein the substitution comprises at least one amino acid chosen from the group consisting of V251E, K287Q, E289K, D324N and D330N, or a combination of two or more thereof.

38. Polypeptide according to claim 36, wherein the insertion:
comprises 3 to 8 amino acids, preferably 3 to 7 amino acids, more preferably 3 to 6 amino acids, even more preferably 3 to 5 amino acids, still even more preferably 3 to 4 amino acids, most preferably 3 amino acids;
said insertion being preferably between positions 324 and 328 (inclusive), more preferably 325 and 327 (inclusive), most preferably between positions 326 and 327 (inclusive) of SEQ ID NO: 1;
preferably comprises at least one glycine; and/or
preferably comprises at least one serine, more preferably at least two serines.

39. Polypeptide according to claim 36, wherein the modification comprises insertion of the contiguous amino acid residues Gly-Ser-Ser (G-S-S), said insertion preferably corresponding to amino acid residues at positions 327-329 of SEQ ID NO:2 between positions 326 and 327 of SEQ ID NO: 1.

40. Polypeptide according to claim 36, wherein the modification comprises amino acid substitutions V251E, K287Q, E289K, D324N and D330N, and insertions 326aG, 326bS and 326cS.

41. Polypeptide according to claim 36, wherein the modification further comprises a substitution of one or more amino acids at the positions chosen from 325, 335 and 343 of SEQ ID NO: 1 by another amino acid, preferably chosen from the group, consisting of glycine (Gly; G), alanine (Ala; A), valine (Val; V), leucine (Leu; L), isoleucine (Ile; I), methionine (Met; M), phenylalanine (Phe: F) tryptophan (Trp; W), serine (Ser; S), threonine (Thr; T), cysteine (Cys; C), tyrosine (Tyr; Y), asparagine (Asn; N), glutamine (Gln; Q), aspartic acid (Asp; D), glutamic acid (Glu; E), lysine (Lys; K), proline (Pro: P), histidine (His; H) and arginine (Arg; R), more preferably the amino acid at position 325 is alanine, more preferably the amino acid at position 335 is tyrosine or more preferably amino acid at position 343 is valine.

42. Polypeptide according to claim 36, wherein the modification further comprises introduction of at least one glycosylation site in the said carbohydrate recognition domain, preferably
of at least two glycosylation sites in the said carbohydrate recognition domain,
wherein the said introduced glycosylation site preferably:
has the amino acid sequence Asn-X-Ser or Asn-X-Thr, wherein X can be any amino acid except proline; and wherein said introduced glycosylation site is preferably glycosylated with a carbohydrate, said carbohydrate preferably comprises at least one sialic acid residue, preferably at least one terminal sialic acid residue, said sialic acid residue is preferably linked to the carbohydrate by alpha (2, 3)-linkage or alpha (2, 6)-linkage, or a mixture thereof; and/or
is introduced at a position between amino acid 240 and 260, preferably between 246 and 253, and/or between 267 and 298, preferably between 272 and 290, and/or between 304 and 331, preferably between 331 and 344, an amino acid sequence of said carbohydrate recognition domain.

43. Polypeptide according to claim 36, wherein the polypeptide:
comprises an N-terminal domain comprising cysteine residues and/or a collagen-like domain characterised by repetitive Gly-Xaa-Yaa sequences and/or a neck-domain;
is preferably in the form of a multimer, preferably a trimer, more preferably hexamer, even more preferably nonamer more preferably a dodecamer, most preferably a multidodecamer; and/or
is a non-natural polypeptide.

44. Polypeptide according to claim 36 for use in the treatment of a disease or the prophylactic treatment of an animal body, preferably a mammalian body, more preferably a human body.

45. Polypeptide for use according to claim 44 in the treatment of a respiratory disease, whereby the said respiratory disease is chosen from the group consisting of viral infections, bacterial infections, fungal infections and inflammatory disorders.

46. Polypeptide for use according to claim 45 wherein:
the viral infection to be treated is chosen from the group consisting of the influenza A virus, influenza B virus, or influenza C virus, Ebola virus, rhinoviruses, coronviruses, parainfluenza viruses, adenoviruses, herpesviruses, respiratory syncytial virus, metapneumovirus and enteroviruses vaccinia virus;
the viral infection to be treated is a virus chosen from the group consisting of the genera influenza A virus, influenza B virus or influenza C virus, wherein genus influenza A virus (IAV) comprises subtypes H1N1, H2N2, H3N2, H5N1, H7N7, H1N2, H9N2, H7N2H7N3, H10N7, H7N9;
the bacterial infection to be treated is chosen from the group consisting of Bacillus anthracis; Bordetella pertussis; Borrelia burgdorferi; Brucella abortus; Brucella canis; Brucella melitensis; Brucella suis; Campylobacter jejuni; Chlamydia pneumonia; Chlamydia trachomatis; Chlamydophila psittaci; Clostridium botulinum; Clostridium difficile; Clostridium perfringens Corynebacterium diphtheria; Enterococcus faecalis and Enterococcus faecium; Escherichia coli sp. in particular, Enterotoxigenic Escherichia coli (ETEC), Enteropathogenic E. coli, E. coli O157:H7; Francisella tularensis; Helicobacter pylori; Haemophilus influenza; Klebsiella pneumonia; Legionella pneumophila; Leptospira interrogans; Listeria monocytogenes; Mycobacterium leprae; Mycobacterium tuberculosis; Neisseria gonorrhoeae; Neisseria meningitides; Pseudomonas aeruginosa; Salmonella enteritidis; Salmonella typhimurium; Shigella sonnei; and Staphylococcus aureus; Streptococcus pneumoniae;
the fungal disease to be treated is chosen from the group consisting of aspergillosis, blastomycosis, candidiasis, coccidioidomycosis, cryptococcosis, histoplasmosis, mucormycosis, pneumocystis pneumonia, and sporotrichosis; or
the inflammatory disorder to be treated is chosen from the group consisting of asthma, autoimmune neutropenia, acute respiratory distress syndrome (ARDS), bronchopulmonary dysplasia (BPD), chronic obstructive pulmonary disorder (COPD), cystic fibrosis, emphysema, hay fever, and sinusitis.

47. Polypeptide for use according to claim 44, wherein the disease to be treated is chosen from the group consisting of arthritis, eczema, Coeliac disease, Crohn's disease, psoriasis, septic shock syndrome, toxic shock syndrome.

48. Polypeptide according to claim 36 for use in the treatment of influenza A virus (IAV) in combination with an existing antiviral agent chosen from the group consisting of neuraminidase inhibitors and adamantanes, wherein the neuraminidase inhibitor is chosen from the group consisting of Laninamivir, Oseltamivir, Zanamivir and Peramivir and the adamantane is chosen from the group consisting of rimantadine and adamantine.

49. Nucleic acid comprising a sequence of nucleotides encoding a polypeptide according to claim 36.

50. Method for obtaining a polypeptide according to claim 36 wherein said method comprises the step of introducing an amino acid mutation in the hSP-D carbohydrate recognition domain, and optionally comprises a further step of
introducing a glycosylation site at a non-glycosylation site in the said carbohydrate recognition domain, wherein said method comprises the steps of a) expressing a nucleic acid encoding a polypeptide according to the invention in a host organism under conditions that the said polypeptide is formed and b) isolating the said polypeptide.
